(19) United States
(12) Patent Application Publication
(10) Pub. No.: US 2025/0255460 A1
(43) Pub. Date: Aug. 14, 2025

(54) MEDICAL SUPPORT DEVICE, ENDOSCOPE, MEDICAL SUPPORT METHOD, AND PROGRAM

(71) Applicant: FUJIFILM Corporation, Tokyo (JP)

(72) Inventor: Misaki GOTO, Kanagawa (JP)

(73) Assignee: FUJIFILM Corporation, Tokyo (JP)

(21) Appl. No.: 19/195,805

(22) Filed: May 1, 2025

Related U.S. Application Data
(63) Continuation of application No. PCT/JP2023/039521, filed on Nov. 1, 2023.

(30) Foreign Application Priority Data
Nov. 4, 2022 (JP) 2022-177609

(51) Int. Cl. A61B 1/00; G06T 7/00; G06T 15/00

(52) U.S. Cl. CPC A61B 1/000094; A61B 1/00045; G06T 7/0012; G06T 15/005; G06T 2207/10068; G06T 2207/30028; G06T 2215/16

(57) ABSTRACT

A medical support device includes a processor. The processor is configured to acquire first reference part information capable of three-dimensionally specifying a reference part included in a duodenum based on a captured image obtained by imaging an intestinal wall of the duodenum with an endoscope scope; acquire second reference part information related to the reference part and direction information related to a running direction of a duct leading to the duodenum from volume data; generate a three-dimensional running direction image in which the running direction is imaged in accordance with the captured image based on the direction information adjusted by matching the first reference part information with the second reference part information; display the captured image on a screen; and display the three-dimensional running direction image in the captured image.

### CROSS-REFERENCE TO RELATED APPLICATIONS

This application is a continuation application of International Application No. PCT/JP2023/039521, filed Nov. 1, 2023, the disclosure of which is incorporated herein by reference in its entirety. Further, this application claims priority from Japanese Patent Application No. 2022-177609, filed Nov. 4, 2022, the disclosure of which is incorporated herein by reference in its entirety.

### BACKGROUND

### 1. Technical Field

The technology of the present disclosure relates to a medical support device, an endoscope, a medical support method, and a program.

### 2. Related Art

JP2022-105685A discloses an endoscope processor comprising an endoscopic image acquisition unit, a virtual endoscopic image acquisition unit, a virtual endoscopic image reconstruction unit, and a diagnosis support information output unit.

In the endoscope processor described in JP2022-105685A, the endoscopic image acquisition unit acquires an endoscopic image of a patient from an endoscope. The virtual endoscopic image acquisition unit acquires a virtual endoscopic image reconstructed based on a three-dimensional medical image of the patient captured in advance. The virtual endoscopic image reconstruction unit reconstructs a corrected virtual endoscopic image that most matches an endoscopic image based on a rate of match between a virtual endoscopic image acquired by the virtual endoscopic image acquisition unit and the endoscopic image acquired by the endoscopic image acquisition unit. The diagnosis support information output unit associates each pixel of the endoscopic image acquired by the endoscopic image acquisition unit with a distance image obtained from the corrected virtual endoscopic image reconstructed by the virtual endoscopic image reconstruction unit, and outputs diagnosis support information based on a feature parameter corrected accordingly.

### SUMMARY

One embodiment according to the technology of the present disclosure provides a medical support device, an endoscope, a medical support method, and a program, which enable a user who observes a captured image to visually recognize a running direction of a duct leading to a duodenum through the captured image.

A first aspect according to the technology of the present disclosure is a medical support device comprising a processor, in which the processor is configured to acquire first reference part information capable of three-dimensionally specifying a reference part included in a duodenum based on a captured image obtained by imaging an intestinal wall of the duodenum with an endoscope scope; acquire second reference part information related to the reference part and direction information related to a running direction of a duct leading to the duodenum from volume data; generate a running direction image in which the running direction is imaged in accordance with the captured image based on the direction information adjusted by matching the first reference part information with the second reference part information; display the captured image on a screen; and display the running direction image in the captured image.

A second aspect according to the technology of the present disclosure is the medical support device according to the first aspect, in which the first reference part information is a first image region in which the reference part is represented as a three-dimensional image, the second reference part information is a second image region in which the reference part is represented as a three-dimensional image, and the processor is configured to generate the running direction image based on the direction information adjusted by performing registration between the first image region and the second image region.

A third aspect according to the technology of the present disclosure is the medical support device according to the first aspect or the second aspect, in which the processor is configured to generate a duodenum image in which the duodenum is represented as a three-dimensional image based on the captured image; and acquire the first reference part information from the duodenum image.

A fourth aspect according to the technology of the present disclosure is the medical support device according to the third aspect, in which the duodenum image is a three-dimensional image generated based on a plurality of distance images.

A fifth aspect according to the technology of the present disclosure is the medical support device according to the fourth aspect, in which, each time the captured image is updated in units of a designated number of frames, the processor is configured to generate the duodenum image based on the updated captured image; generate the running direction image based on the direction information each time the duodenum image is generated; display the updated captured image on the screen; and display the generated running direction image in the captured image each time the running direction image is generated.

A sixth aspect according to the technology of the present disclosure is the medical support device according to any one of the first to fifth aspects, in which the direction information is a three-dimensional direction image in which the running direction is represented as a three-dimensional image, and the running direction image is an image based on a two-dimensional image obtained by projecting the three-dimensional direction image onto the captured image.

A seventh aspect according to the technology of the present disclosure is the medical support device according to any one of the first to sixth aspects, in which the captured image includes a papilla image showing a duodenal papilla, and the running direction image is displayed in association with the papilla image.

An eighth aspect according to the technology of the present disclosure is the medical support device according to the seventh aspect, in which the duct leads to an opening in the duodenal papilla, and the running direction image is an image showing a first direction along the duct with the opening as a base point.

A ninth aspect according to the technology of the present disclosure is the medical support device according to the eighth aspect, in which the first direction is a direction corresponding to an insertion direction of a medical instrument to be inserted into the duct.

A tenth aspect according to the technology of the present disclosure is the medical support device according to any one of the first to ninth aspects, in which the reference part is a duodenal papilla, a medical marker, and/or a fold.

An eleventh aspect according to the technology of the present disclosure is the medical support device according to any one of the first to tenth aspects, in which the processor is configured to adjust a scale of the direction information based on a distance from the endoscope scope to the intestinal wall and on the volume data.

A twelfth aspect according to the technology of the present disclosure is the medical support device according to any one of the first to tenth aspects, in which the duct is a bile duct and/or a pancreatic duct.

A thirteenth aspect according to the technology of the present disclosure is the medical support device according to any one of the first to twelfth aspects, in which the running direction image displayed in the captured image is updated in real time.

A fourteenth aspect of the technology of the present disclosure is an endoscope comprising the medical support device according to any one of the first to thirteenth aspects; and the endoscope scope.

A fifteenth aspect according to the technology of the present disclosure is a medical support method comprising acquiring first reference part information capable of three-dimensionally specifying a reference part included in a duodenum based on a captured image obtained by imaging an intestinal wall of the duodenum with an endoscope scope; acquiring second reference part information related to the reference part and direction information related to a running direction of a duct leading to the duodenum from volume data; generating a running direction image in which the running direction is imaged in accordance with the captured image based on the direction information adjusted by matching the first reference part information with the second reference part information; displaying the captured image on a screen; and displaying the running direction image in the captured image.

A sixteenth aspect according to the technology of the present disclosure is a program causing a computer to execute processing comprising acquiring first reference part information capable of three-dimensionally specifying a reference part included in a duodenum based on a captured image obtained by imaging an intestinal wall of the duodenum with an endoscope scope; acquiring second reference part information related to the reference part and direction information related to a running direction of a duct leading to the duodenum from volume data; generating a running direction image in which the running direction is imaged in accordance with the captured image based on the direction information adjusted by matching the first reference part information with the second reference part information; displaying the captured image on a screen; and displaying the running direction image in the captured image.

### BRIEF DESCRIPTION OF THE DRAWINGS

Exemplary embodiments according to the technique of the present disclosure will be described in detail based on the following figures, wherein:

FIG. 1 is a conceptual diagram showing an example of an aspect in which a duodenoscope system is used;

FIG. 2 is a conceptual diagram showing an example of an overall configuration of the duodenoscope system;

FIG. 3 is a block diagram showing an example of a hardware configuration of an electrical system of the duodenoscope system;

FIG. 4 is a conceptual diagram showing an example of aspects of a duodenum, a bile duct, and a pancreatic duct;

FIG. 5 is a block diagram showing an example of functions of main units of a processor included in an endoscope and an example of information stored in an NVM;

FIG. 6 is a conceptual diagram showing an example of a processing content of a first acquisition unit;

FIG. 7 is a conceptual diagram showing an example of a processing content of a second acquisition unit;

FIG. 8 is a conceptual diagram showing an example of a first processing content of an adjustment unit;

FIG. 9 is a conceptual diagram showing an example of a second processing content of the adjustment unit;

FIG. 10 is a conceptual diagram showing an example of a processing content of a synthesis unit;

FIG. 11 is a conceptual diagram showing an example of a first processing content of a third acquisition unit;

FIG. 12 is a conceptual diagram showing an example of a second processing content of the third acquisition unit;

FIG. 13 is a conceptual diagram showing an example of a first processing content of a control unit;

FIG. 14 is a conceptual diagram showing an example of a second processing content of the control unit;

FIG. 15A is a flowchart showing an example of a flow of medical support processing;

FIG. 15B is a continuation of the flowchart shown in FIG. 15A;

FIG. 16 is a conceptual diagram showing a modification example of the processing content of the first acquisition unit;

FIG. 17 is a conceptual diagram showing a modification example of the processing content of the third acquisition unit; and

FIG. 18 is a conceptual diagram showing an example of an aspect in which information is output to a server and/or a printer from an image processing device or a control device via a network.

### DETAILED DESCRIPTION

Hereinafter, examples of embodiments of a medical support device, an endoscope, a medical support method, and a program according to the technology of the present disclosure will be described with reference to the accompanying drawings.

First, terms used in the following description will be described.

CPU is an abbreviation for “central processing unit”. GPU is an abbreviation for “graphics processing unit”. RAM is an abbreviation for “random-access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. AI is an abbreviation for “artificial intelligence”. BLI is an abbreviation for “blue light imaging”. LCI is an abbreviation for “linked color imaging”. I/F is an abbreviation for “interface”. FIFO is an abbreviation for “first in, first out”. ToF is an abbreviation for “time of flight”. RNN-SLAM is an abbreviation for “recurrent neural network-simultaneous localization and mapping”. VAE is an abbreviation for “variational auto-encoder”. GAN is an abbreviation for “generative adversarial network”. CT is an abbreviation for “computed tomography”. MRI is an abbreviation for “magnetic resonance imaging”. 3D is an abbreviation for “three dimensions”.

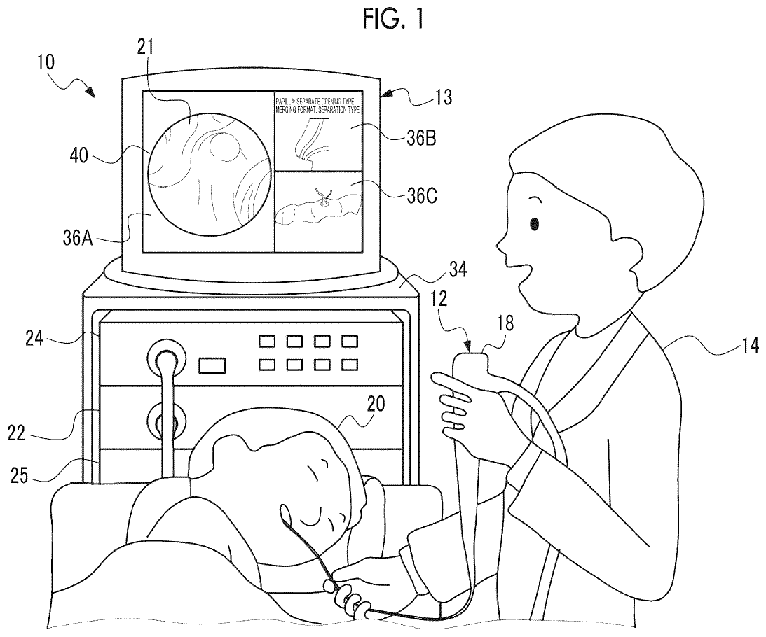

For example, as shown in FIG. 1, a duodenoscope system 10 comprises a duodenoscope 12 and a display device 13. The duodenoscope 12 is used by a doctor 14 in endoscopy. The duodenoscope 12 is an example of the “endoscope” according to the technology of the present disclosure.

The duodenoscope 12 is communicably connected to a communication device (not shown), and information obtained by the duodenoscope 12 is transmitted to the communication device. The communication device receives the information transmitted from the duodenoscope 12 and performs processing using the received information (for example, the processing of recording the information on an electronic medical record or the like).

The duodenoscope 12 comprises a duodenoscope body 18 (in other words, an endoscope scope). The duodenoscope 12 is a device for performing medical care on an observation target 21 (for example, a duodenum) included in a body of a subject 20 (for example, a patient) by using the duodenoscope body 18. The observation target 21 is a target observed by the doctor 14.

The duodenoscope body 18 is inserted into the body of the subject 20. The duodenoscope 12 images the observation target 21 in the body of the subject 20 with respect to the duodenoscope body 18 inserted into the body of the subject 20, and performs various medical treatments on the observation target 21 as necessary.

The duodenoscope 12 images the inside of the body of the subject 20 to acquire an image showing an aspect of the inside of the body and outputs the image. In the present embodiment, the duodenoscope 12 is an endoscope having an optical imaging function of irradiating the inside of the body with light to image the light reflected by the observation target 21.

The duodenoscope 12 comprises a control device 22, a light source device 24, and an image processing device 25. The control device 22, the light source device 24, and the image processing device 25 are installed in a wagon 34. A plurality of tables are provided in the wagon 34 in a vertical direction, and the image processing device 25, the control device 22, and the light source device 24 are installed from a lower table to an upper table. In addition, the display device 13 is installed on the uppermost table in the wagon 34.

The control device 22 controls the entire duodenoscope 12. The image processing device 25 performs various types of image processing on the image obtained by imaging the observation target 21 with the duodenoscope body 18 under the control of the control device 22.

The display device 13 displays various types of information including images. An example of the display device 13 is a liquid-crystal display or an EL display. In addition, a tablet terminal with a display may be used instead of the display device 13 or together with the display device 13.

A plurality of screens are displayed side by side on the display device 13. In the example shown in FIG. 1, screens 36A to 36C are shown as an example of a plurality of screens.

A captured image 40 obtained by the duodenoscope 12 is displayed on the screen 36A. The observation target 21 is captured in the captured image 40. The captured image 40 is an image obtained by imaging the observation target 21 with the duodenoscope body 18 in the body of the subject 20. An example of the observation target 21 includes an intestinal wall of a duodenum. In the following, for convenience of explanation, an intestinal wall image is given as an example of the captured image 40. The intestinal wall image refers to an image obtained by imaging the intestinal wall of the duodenum as the observation target 21. In addition, the duodenum is merely an example, and any region that can be imaged by the duodenoscope 12 may be used. For example, an esophagus or a stomach is given as an example of the region that can be imaged by the duodenoscope 12. The captured image 40 is an example of the “captured image” according to the technology of the present disclosure.

A moving image configured to include a plurality of frames of the captured images 40 in time series is displayed on the screen 36A. That is, the plurality of frames of captured images 40 are displayed in time series on the screen 36A at a predetermined frame rate (for example, several tens of frames/sec). The screen 36A is an example of the “screen” according to the technology of the present disclosure.

The screen 36A is a main screen, while the screen 36B and the screen 36C are sub-screens, and various types of information for supporting the procedure using the duodenoscope 12 by the doctor 14 are displayed on the screen 36B and the screen 36C.

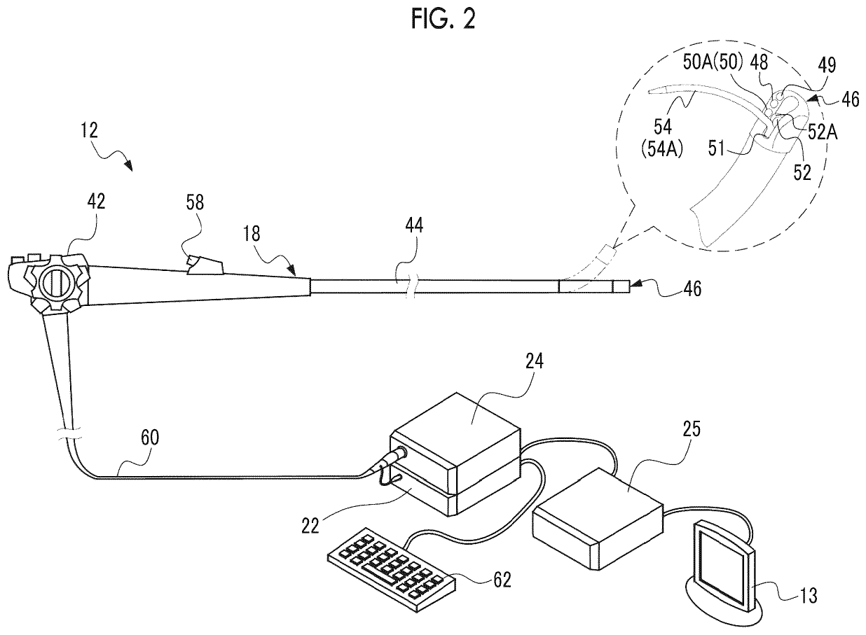

For example, as shown in FIG. 2, the duodenoscope body 18 comprises an operation part 42 and an insertion part 44. The insertion part 44 is partially bent by operating the operation part 42. The insertion part 44 is inserted while being bent according to the shape of the observation target 21 (for example, the shape of the stomach) in response to the operation of the operation part 42 by the doctor 14.

A camera 48, a distance-measuring sensor 49, an illumination device 50, a treatment opening 51, and an elevating mechanism 52 are provided at a distal end part 46 of the insertion part 44. The camera 48 and the illumination device 50 are provided on a side surface of the distal end part 46. That is, the duodenoscope 12 is configured as a side-viewing scope and enables a user to easily observe the intestinal wall of the duodenum.

The camera 48 is a device that acquires the captured image 40 as a medical image by imaging the inside of the body of the subject 20. An example of the camera 48 includes a CMOS camera. However, this is merely an example, and the camera 48 may be other types of cameras such as CCD cameras. The camera 48 is an example of the “endoscope scope” according to the technology of the present disclosure.

The distance-measuring sensor 49 is an optical distance-measuring sensor. For example, the distance-measuring sensor 49 performs distance measurement (that is, measurement of distance) in synchronization with the imaging timing of the camera 48. An example of the distance-measuring sensor 49 includes a device including a ToF camera. The ToF camera is a camera that measures three-dimensional information by a ToF method. Here, although the camera 48 and the distance-measuring sensor 49 are separate from each other, the camera 48 may be equipped with the function of the ToF camera. In this case, the camera 48 can also obtain depth information at the same time as the captured image 40.

The illumination device 50 has an illumination window 50A. The illumination device 50 emits light through the illumination window 50A. Examples of the type of the light emitted from the illumination device 50 include visible light (for example, white light) and invisible light (for example, near-infrared light). In addition, the illumination device 50 emits special light through the illumination window 50A. Examples of the special light include light for BLI and/or light for LCI. The camera 48 images the inside of the body of the subject 20 using an optical method in a state in which the inside of the body of the subject 20 is irradiated with light by the illumination device 50.

The treatment opening 51 is used as a treatment tool protruding port through which a treatment tool 54 protrudes from the distal end part 46, a suction port for suctioning blood, internal excrement, and the like, and a delivery port for delivering a fluid.

The treatment tool 54 protrudes from the treatment opening 51 in response to the operation of the doctor 14. The treatment tool 54 is inserted into the insertion part 44 from a treatment tool insertion port 58. The treatment tool 54 passes through the inside of the insertion part 44 through the treatment tool insertion port 58 and protrudes from the treatment opening 51 into the body of the subject 20. In the example shown in FIG. 2, a cannula 54A protrudes from the treatment opening 51 as the treatment tool 54. The cannula 54A is merely an example of the treatment tool 54, and other examples of the treatment tool 54 include a catheter, a guide wire, a papillotomy knife, and a snare. The treatment tool 54 is an example of the “medical instrument” according to the technology of the present disclosure.

The elevating mechanism 52 changes a protruding direction of the treatment tool 54 protruding from the treatment opening 51. The elevating mechanism 52 comprises a guide 52A, and the guide 52A rises with respect to the protruding direction of the treatment tool 54, so that the protruding direction of the treatment tool 54 is changed along the guide 52A. Accordingly, it is easy to cause the treatment tool 54 to protrude toward the intestinal wall. In the example shown in FIG. 2, the protruding direction of the treatment tool 54 is changed to a direction perpendicular to a traveling direction of the distal end part 46 by the elevating mechanism 52. The elevating mechanism 52 is operated by the doctor 14 using the operation part 42. Accordingly, the degree of change in the protruding direction of the treatment tool 54 is adjusted.

The duodenoscope body 18 is connected to the control device 22 and the light source device 24 via a universal cord 60. A receiving device 62 is connected to the control device 22. In addition, an image processing device 25 is connected to the control device 22. In addition, the display device 13 is connected to the image processing device 25. That is, the control device 22 is connected to the display device 13 via the image processing device 25.

In addition, here, the image processing device 25 is given as an example of an external device for expanding the functions of the control device 22. Therefore, the form example in which the control device 22 and the display device 13 are indirectly connected to each other via the image processing device 25 is given. However, this is merely an example. For example, the display device 13 may be directly connected to the control device 22. In this case, for example, the functions of the image processing device 25 may be provided in the control device 22, or the control device 22 may be provided with a function of directing a server (not shown) to perform the same process as the process (for example, medical support processing which will be described below) performed by the image processing device 25, receiving a processing result of the server, and using the processing result.

The receiving device 62 receives an instruction from a user (for example, the doctor 14) and outputs the received instruction to the control device 22 as an electric signal. Examples of the receiving device 62 include a keyboard, a mouse, a touch panel, a foot switch, and a microphone.

The control device 22 controls the light source device 24, transmits and receives various signals with the camera 48, or transmits and receives various signals with the image processing device 25.

The light source device 24 emits light under the control of the control device 22 and supplies the light to the illumination device 50. A light guide is provided in the illumination device 50, and the light supplied from the light source device 24 is emitted from the illumination window 50A via the light guide. The control device 22 causes the camera 48 to execute the imaging, acquires the captured image 40 (see FIG. 1) from the camera 48, and outputs the captured image 40 to a predetermined output destination (for example, the image processing device 25).

The image processing device 25 performs various types of image processing on the captured image 40 input from the control device 22. The image processing device 25 outputs the captured image 40 subjected to various types of image processing to a predetermined output destination (for example, the display device 13).

In addition, here, although the form example in which the captured image 40 output from the control device 22 is output to the display device 13 via the image processing device 25 has been described, this is merely an example. The control device 22 and the display device 13 may be connected to each other, and the captured image 40 subjected to the image processing by the image processing device 25 may be displayed on the display device 13 via the control device 22.

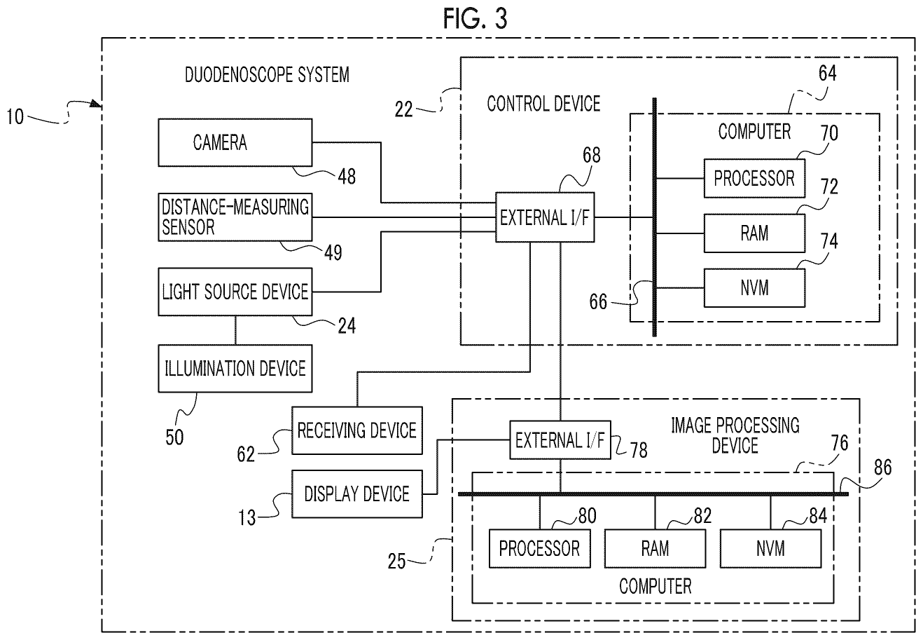

As shown in FIG. 3 as an example, the control device 22 comprises a computer 64, a bus 66, and an external I/F 68. The computer 64 comprises a processor 70, a RAM 72, and an NVM 74. The processor 70, the RAM 72, the NVM 74, and the external I/F 68 are connected to the bus 66.

For example, the processor 70 includes a CPU and a GPU and controls the entire control device 22. The GPU operates under the control of the CPU and is in charge of, for example, executing various processing operations of a graphics system and performing calculation using a neural network. In addition, the processor 70 may be one or more CPUs with which the functions of the GPU have been integrated or may be one or more CPUs with which the functions of the GPU have not been integrated.

The RAM 72 is a memory that temporarily stores information and is used as a work memory by the processor 70. The NVM 74 is a non-volatile storage device that stores, for example, various programs and various parameters. An example of the NVM 74 includes a flash memory (for example, an EEPROM and/or an SSD). In addition, the flash memory is merely an example and may be other non-volatile storage devices, such as HDDs, or a combination of two or more types of non-volatile storage devices.

The external I/F 68 controls the transmission and the reception of various types of information between one or more devices (hereinafter also referred to as “first external devices”) present outside the control device 22 and the processor 70. An example of the external I/F 68 is a USB interface.

The camera 48 is connected to the external I/F 68 as one of the first external devices, and the external I/F 68 controls the transmission and the reception of various types of information between the camera 48 and the processor 70. The processor 70 controls the camera 48 via the external I/F 68. In addition, the processor 70 acquires, via the external I/F 68, the captured image 40 (see FIG. 1) obtained by imaging the inside of the body of the subject 20 via the camera 48.

The distance-measuring sensor 49 is connected to the external I/F 68 as one of the first external devices, and the external I/F 68 controls the transmission and the reception of various types of information between the distance-measuring sensor 49 and the processor 70. The processor 70 controls the distance-measuring sensor 49 and acquires a result (for example, a distance or three-dimensional information measured by the distance-measuring sensor 49) of distance measurement performed by the distance-measuring sensor 49.

The light source device 24 is connected to the external I/F 68 as one of the first external devices, and the external I/F 68 controls the transmission and the reception of various types of information between the light source device 24 and the processor 70. The light source device 24 supplies light to the illumination device 50 under the control of the processor 70. The illumination device 50 performs irradiation with the light supplied from the light source device 24.

The receiving device 62 is connected to the external I/F 68 as one of the first external devices, and the processor 70 acquires the instruction received by the receiving device 62 via the external I/F 68 and executes processing in response to the acquired instruction.

The image processing device 25 comprises a computer 76 and an external I/F 78. The computer 76 comprises a processor 80, a RAM 82, and an NVM 84. The processor 80, the RAM 82, the NVM 84, and the external I/F 78 are connected to a bus 86. Here, the image processing device 25 is an example of the “medical support device” according to the technology of the present disclosure, the computer 76 is an example of the “computer” according to the technology of the present disclosure, and the processor 80 is an example of the “processor” according to the technology of the present disclosure.

In addition, since a hardware configuration (that is, the processor 80, the RAM 82, and the NVM 84) of the computer 76 is essentially the same as a hardware configuration of the computer 64, the description of the hardware configuration of the computer 76 will be omitted here.

The external I/F 78 controls the transmission and the reception of various types of information between one or more devices (hereinafter, also referred to as “second external devices”) outside the image processing device 25 and the processor 80. An example of the external I/F 78 is a USB interface.

The control device 22 is connected to the external I/F 78 as one of the second external devices. In the example shown in FIG. 3, the external I/F 68 of the control device 22 is connected to the external I/F 78. The external I/F 78 controls the transmission and the reception of various types of information between the processor 80 of the image processing device 25 and the processor 70 of the control device 22. For example, the processor 80 acquires the captured image 40 (see FIG. 1) from the processor 70 of the control device 22 via the external I/Fs 68 and 78 and performs various types of image processing on the acquired captured image 40.

The display device 13 is connected to the external I/F 78 as one of the second external devices. The processor 80 controls the display device 13 via the external I/F 78 such that various types of information (for example, the captured image 40 subjected to various types of image processing) are displayed on the display device 13.

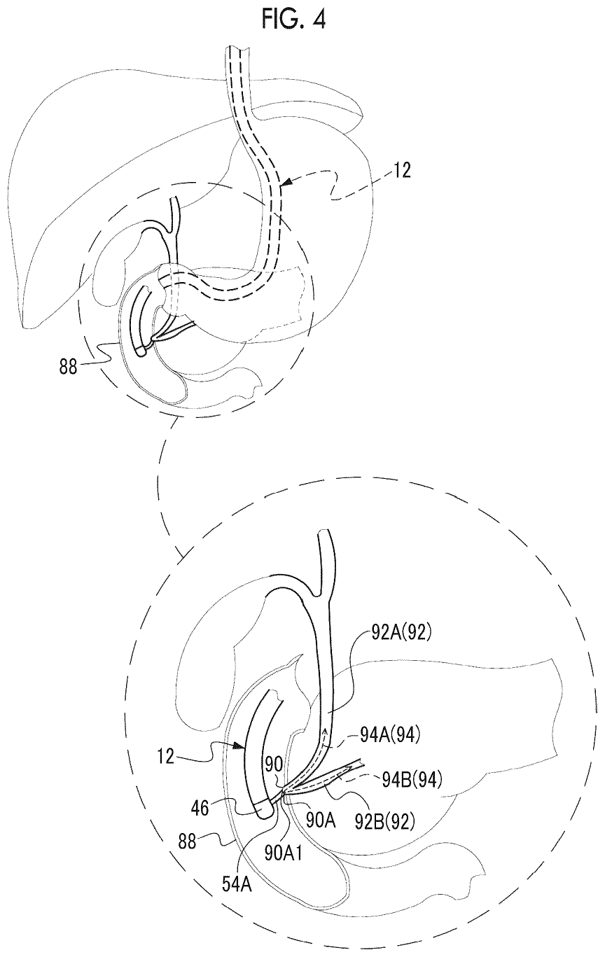

As one of treatments for the duodenum using the duodenoscope 12, a treatment called endoscopic retrograde cholangio-pancreatography (ERCP) examination is known. As shown in FIG. 4 as an example, in the ERCP examination, for example, first, the duodenoscope 12 is inserted into a duodenum 88 through the esophagus and the stomach. In this case, an insertion state of the duodenoscope 12 may be checked by using an X-ray image obtained by X-ray imaging. Then, the distal end part 46 of the duodenoscope 12 reaches the vicinity of a duodenal papilla 90 (hereinafter, also simply referred to as a “papilla 90”) present in the intestinal wall of the duodenum 88.

In the ERCP examination, for example, a cannula 54A, which is a type of treatment tool 54, is inserted into the papilla 90 from the duodenum 88 side. Here, the papilla 90 is a part that protrudes from the intestinal wall of the duodenum 88. An end part of one or more ducts 92 leading to internal organs (for example, a gallbladder and a pancreas), that is, an opening 90A1 leading to the duct 92 is present at a papillary protuberance 90A, which is a distal end part of the papilla 90. In other words, the duct 92 leads to the opening 90A1 present in the papillary protuberance 90A.

Examples of one or more ducts 92 include a bile duct 92A and a pancreatic duct 92B. The opening 90A1 may be individually present for each of the bile duct 92A and the pancreatic duct 92B, or may be present in common to the bile duct 92A and the pancreatic duct 92B. The bile duct 92A is an example of the “bile duct” according to the technology of the present disclosure, and the pancreatic duct 92B is an example of the “pancreatic duct” according to the technology of the present disclosure. In the following, for convenience of description, in a case where it is not necessary to distinguish between the bile duct 92A and the pancreatic duct 92B, the bile duct 92A and the pancreatic duct 92B will be referred to as the “duct 92”.

In the ERCP examination, the X-ray imaging is performed in a state in which a contrast agent is injected into the duct 92 through the opening 90A1. Here, in a case in which the doctor 14 inserts the cannula 54A into the duct 92, it is necessary to accurately grasp a running direction 94 of the duct 92. In particular, since the running direction 94 in the vicinity of the opening 90A1 substantially matches the insertion direction of the cannula 54A with respect to the opening 90A1, it is very important for the doctor 14 to visually grasp the running direction 94 in the vicinity of the opening 90A1.

Examples of the running direction 94 of the duct 92 include a running direction 94A of the bile duct 92A and a running direction 94B of the pancreatic duct 92B. It is effective for the doctor 14 to visually grasp the running direction 94A in a case in which the cannula 54A is inserted into the bile duct 92A, and it is effective for the doctor 14 to visually grasp the running direction 94B in a case in which the cannula 54A is inserted into the pancreatic duct 92B. Here, although an example in which the cannula 54A is inserted into the opening 90A1 has been described, even in a case where a catheter or a guide wire is inserted into the duct 92 as the treatment tool 54, or even in a case where a papillotomy knife is brought into contact with the opening 90A1 as the treatment tool 54, it is very effective for the doctor 14 to visually grasp the running direction 94.

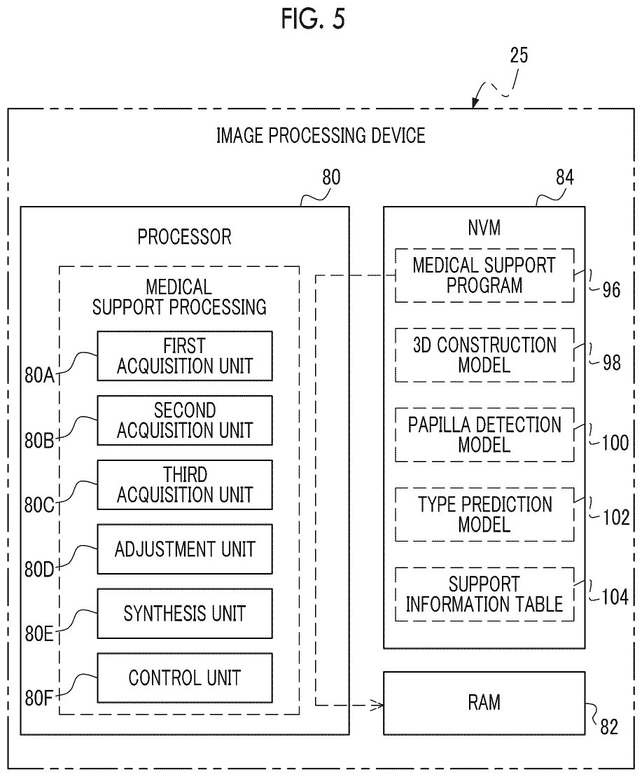

Thus, in view of these circumstances, in the present embodiment, for example, as shown in FIG. 5, the processor 80 of the image processing device 25 performs the medical support processing.

A medical support program 96 is stored in the NVM 84. The medical support program 96 is an example of the “program” according to the technology of the present disclosure. The processor 80 reads out the medical support program 96 from the NVM 84 and executes the read medical support program 96 on the RAM 82 to perform the medical support processing. The medical support processing is realized by the processor 80 operating as a first acquisition unit 80A, a second acquisition unit 80B, a third acquisition unit 80C, an adjustment unit 80D, a synthesis unit 80E, and a control unit 80F according to the medical support program 96 executed on the RAM 82.

A 3D construction model 98, a papilla detection model 100, a type prediction model 102, and a support information table 104 are stored in the NVM 84. As will be described in detail below, the 3D construction model 98 is used by the first acquisition unit 80A, the papilla detection model 100 is used by the adjustment unit 80D, and the type prediction model 102 and the support information table 104 are used by the control unit 80F.

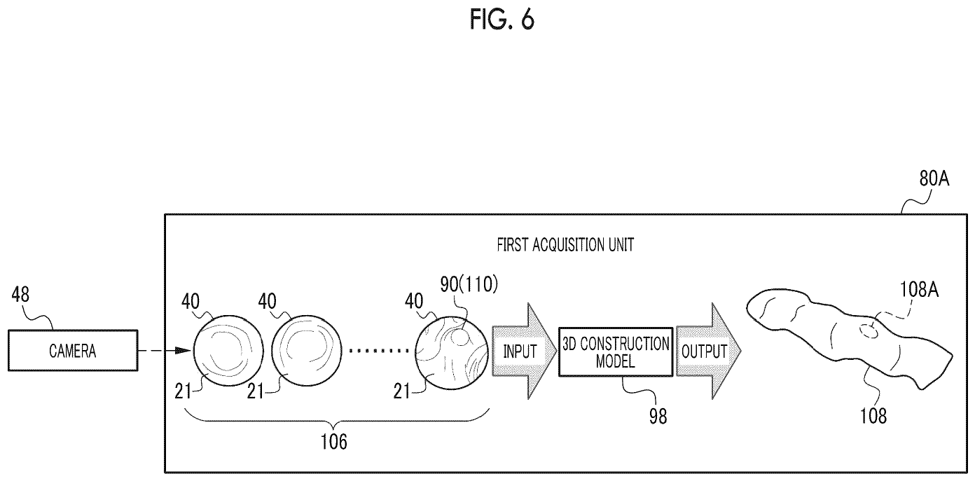

As shown in FIG. 6 as an example, the first acquisition unit 80A acquires a first duodenum image 108 based on a time-series image group 106. The acquisition of the first duodenum image 108 is realized, for example, by generating the first duodenum image 108 by using the 3D construction model 98.

In order to realize the generation of the first duodenum image 108, first, the first acquisition unit 80A acquires the captured image 40 generated by being captured by the camera 48 according to an imaging frame rate (for example, several tens of frames/second) in units of one frame from the camera 48. The units of one frame are an example of the “units of a designated number of frames” according to the technology of the present disclosure. In addition, here, although the units of one frame are exemplified, this is merely an example, and units of a designated number of frames including two or more of frames may be used.

The first acquisition unit 80A holds the time-series image group 106. The time-series image group 106 is a plurality of frames of the captured images 40 in a time series in which the observation target 21 is captured. The time-series image group 106 includes, for example, a predetermined number of frames (for example, a predetermined number of frames within a range of several tens to several hundreds of frames) of captured images 40. In addition, the papilla 90 is captured in one or more frames of the captured images 40 among the predetermined number of frames of captured images 40. That is, one or more frames of the captured images 40 include a papilla image 110 showing the papilla 90.

The first acquisition unit 80A updates the time-series image group 106 using a FIFO method each time the captured image 40 is acquired from the camera 48.

Here, a form example in which the time-series image group 106 is held and updated by the first image acquisition unit 80A has been described, but this is merely an example. For example, the time-series image group 106 may be held and updated in a memory, such as the RAM 82, which is connected to the processor 80.

The 3D construction model 98 is a generation model using a neural network, and generates the first duodenum image 108, which is a three-dimensional image, based on the time-series image group 106. The 3D construction model 98 is a trained model obtained by performing machine learning using a plurality of images corresponding to the time-series image group 106 as training data on the neural network (for example, a recurrent neural network). An example of the 3D construction model 98 includes RNN-SLAM. In addition, the RNN-SLAM is merely an example, and the 3D construction model 98 may be an auto-encoder such as a VAE or a GAN, or any other trained generative network capable of generating the first duodenum image 108.

The first acquisition unit 80A inputs the time-series image group 106 to the 3D construction model 98. In response to this, the 3D construction model 98 generates and outputs the first duodenum image 108 based on the time-series image group 106. The first acquisition unit 80A acquires the first duodenum image 108 output from the 3D construction model 98.

The first duodenum image 108 includes a first papilla region 108A. The first papilla region 108A is information capable of three-dimensionally specifying the papilla 90 included in the duodenum 88. In the example shown in FIG. 6, an image region in which the papilla 90 is represented as a three-dimensional image is shown as an example of the first papilla region 108A. The first duodenum image 108 is generated each time the time-series image group 106 is updated by the first acquisition unit 80A by acquiring the captured image 40 in units of one frame.

In the present embodiment, the papilla 90 is an example of the “reference part” and the “duodenal papilla” according to the technology of the present disclosure. The first papilla region 108A is an example of the “first reference part information” and the “first image region” according to the technology of the present disclosure.

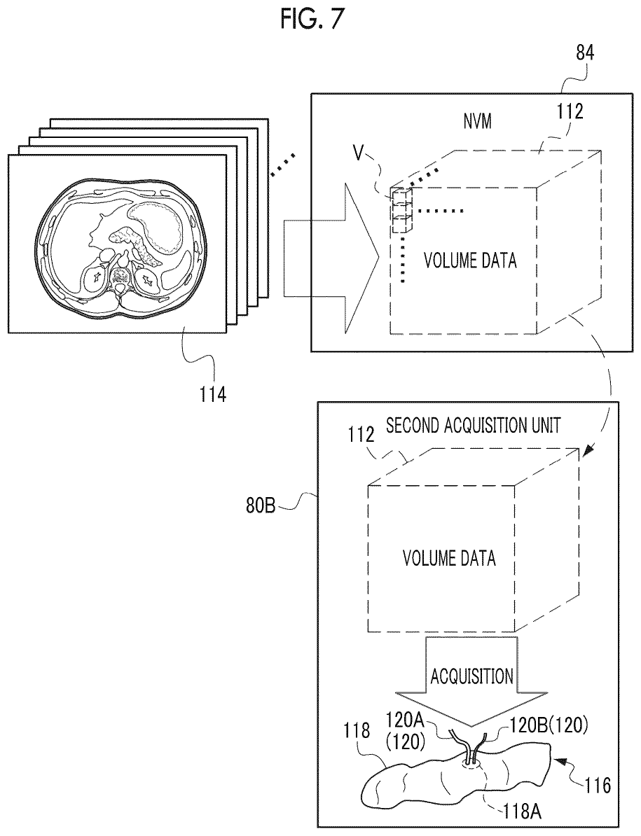

As shown in FIG. 7 as an example, volume data 112 is stored in the NVM 84. The volume data 112 is an image obtained by stacking a plurality of two-dimensional slice images 114 obtained by imaging the subject 20 according to the modality and dividing the stacked images into voxels V. Examples of the modality include a CT apparatus. The CT apparatus is merely an example, and other examples of the modality include an MRI apparatus and an ultrasound diagnostic device. The position of each of all the voxels V that define the three-dimensional image is specified by three-dimensional coordinates.

The second acquisition unit 80B acquires information related to an observation target part from the volume data 112. In the example shown in FIG. 7, an observation target part image 116, which is a three-dimensional image showing the observation target part, is shown as the information related to the observation target part. That is, the second acquisition unit 80B acquires the observation target part image 116 as the information related to the observation target part from the volume data 112.

The observation target part is the duodenum 88, including the papilla 90, and the duct 92 (see FIG. 4). The observation target part (that is, the observation target part image 116 acquired from the volume data 112 by the second acquisition unit 80B) is selected, for example, according to an instruction received by the receiving device 62.

The observation target part image 116 acquired from the volume data 112 by the second acquisition unit 80B includes a second duodenum image 118 and a duct image 120. The second duodenum image 118 is a three-dimensional image showing the duodenum 88. The second duodenum image 118 includes a second papilla region 118A. The second papilla region 118A is an image region in which the papilla 90 is represented as a three-dimensional image. The duct image 120 is an image region in which the duct 92 is represented as a three-dimensional image. The duct image 120 includes a bile duct image 120A and a pancreatic duct image 120B. The bile duct image 120A is an image region in which the bile duct 92A is represented as a three-dimensional image, and the pancreatic duct image 120B is an image region in which the pancreatic duct 92B is represented as a three-dimensional image. In the present embodiment, the second papilla region 118A is an example of the “second reference part information” and the “second image region” according to the technology of the present disclosure.

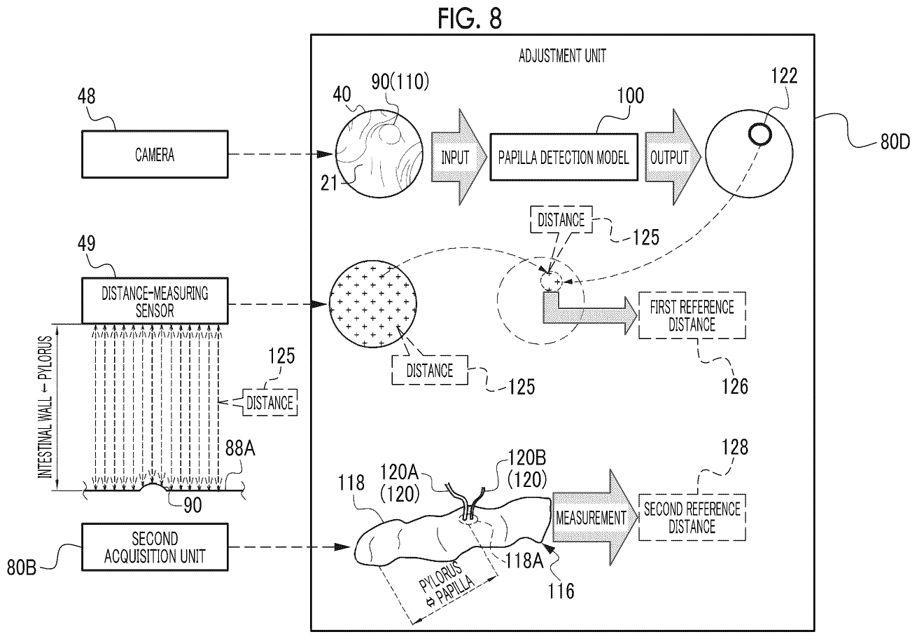

As shown in FIG. 8 as an example, the adjustment unit 80D detects the papilla image 110 in the captured image 40 by performing object detection processing using the papilla detection model 100 on the captured image 40. The papilla detection model 100 is a trained model for object detection using an AI method and is optimized by performing machine learning using first training data on a neural network. The first training data is a plurality of data items (that is, data corresponding to a plurality of frames) in which first example data and first correct answer data have been associated with each other. The first example data is an image corresponding to the captured image 40. The first correct answer data refers to correct answer data (that is, an annotation) for the first example data. An example of the first correct answer data includes an annotation capable of specifying an image region in which the papilla 90 is captured.

The adjustment unit 80D acquires the captured image 40 from the camera 48 and inputs the acquired captured image 40 to the papilla detection model 100. Accordingly, the papilla detection model 100 detects the papilla image 110 from the input captured image 40 and outputs specific information 122 (for example, a plurality of coordinates indicating the position of the papilla image 110 in the captured image 40) capable of specifying the detected papilla image 110. The adjustment unit 80D acquires the specific information 122 output from the papilla detection model 100.

The adjustment unit 80D acquires a distance 125 measured by the distance-measuring sensor 49 in synchronization with the imaging performed by the camera 48 in order to obtain the captured image 40 input to the papilla detection model 100. The distance 125 refers to, for example, a distance from a reference position (here, as an example, the position of an imaging surface of the camera 48 that has performed imaging in order to obtain the captured image 40 input to the papilla detection model 100) to an intestinal wall 88A including the papilla 90 in the duodenum 88. In the example shown in FIG. 8, a pylorus is exemplified as an example of the reference position. The distance-measuring sensor 49 measures a plurality of distances 125 from the reference position to a plurality of points in the intestinal wall 88A.

The adjustment unit 80D calculates a first reference distance 126, which is a distance to the papilla 90 indicated by the papilla image 110 specified by the specific information 122. For example, in a case in which a plurality of measurement points are present in the papilla 90 indicated by the papilla image 110 specified by the specific information 122, an average value of the plurality of distances 125 for the plurality of measurement points is calculated as the first reference distance 126. In addition, here, although the average value of the plurality of distances 125 for the plurality of measurement points in the papilla 90 is shown, a statistical value such as a median value, a most frequent value, or a maximum value of the plurality of distances 125 for the plurality of measurement points in the papilla 90 may be used.

The adjustment unit 80D measures, as a second reference distance 128, a distance from a reference position (here, as an example, the pylorus) to the papilla 90 indicated by the second papilla region 118A, using the second duodenum image 118 included in the observation target part image 116 acquired by the second acquisition unit 80B.

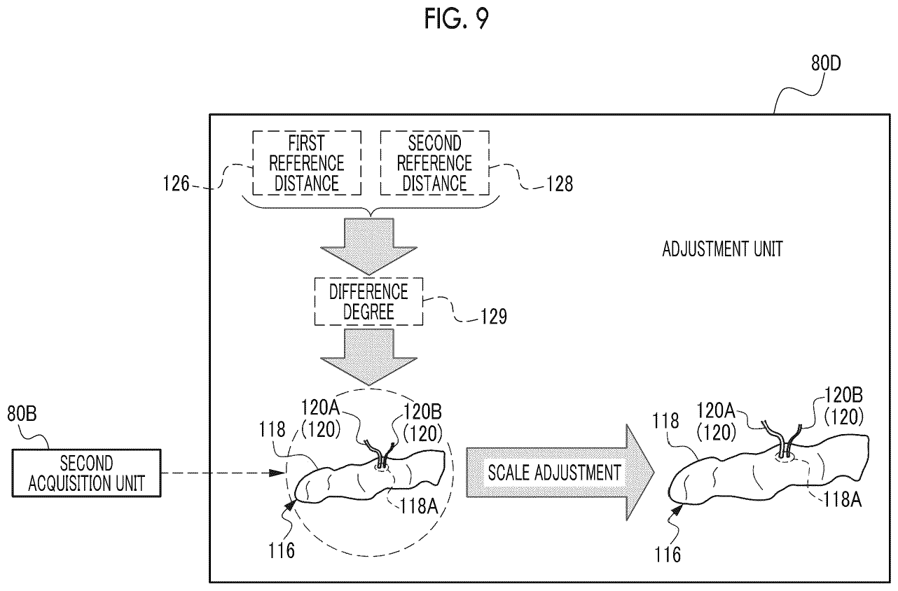

As shown in FIG. 9 as an example, the adjustment unit 80D calculates a difference degree 129 between the first reference distance 126 and the second reference distance 128. Examples of the difference degree 129 include a ratio of the second reference distance 128 to the first reference distance 126, an absolute value of a difference between the first reference distance 126 and the second reference distance 128, or the like.

The adjustment unit 80D adjusts the scale of the observation target part image 116 acquired by the second acquisition unit 80B with reference to the difference degree 129. For example, the adjustment unit 80D adjusts the scale of the observation target part image 116 by using the difference degree 129 itself as the magnification, or adjusts the scale of the observation target part image 116 by the magnification calculated based on the difference degree 129. An example of the magnification calculated based on the difference degree 129 includes a magnification derived from a calculation expression in which the difference degree 129 is set as an independent variable and the magnification is set as a dependent variable.

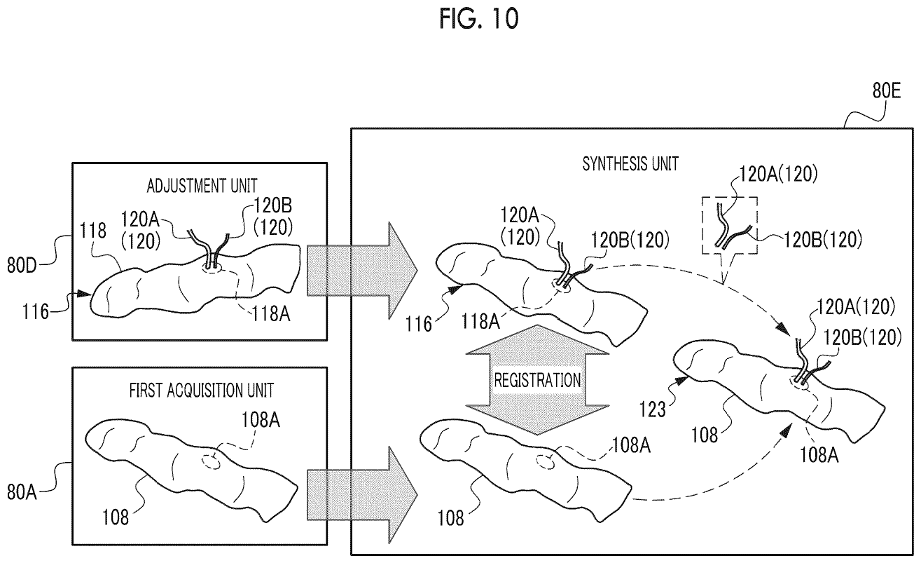

As shown in FIG. 10 as an example, the synthesis unit 80E generates a synthesis image 123 based on the first duodenum image 108 acquired by the first acquisition unit 80A and on the observation target part image 116 of which the scale is adjusted by the adjustment unit 80D. In order to generate the synthesis image 123, first, the synthesis unit 80E adjusts the duct image 120 by matching the first papilla region 108A included in the first duodenum image 108 acquired by the first acquisition unit 80A with the second papilla region 118A included in the observation target part image 116 of which the scale is adjusted by the adjustment unit 80D. For example, the synthesis unit 80E performs registration between the observation target part image 116 and the first duodenum image 108 with reference to the first papilla region 108A and the second papilla region 118A. That is, the observation target part image 116 is registered with the first duodenum image 108 such that the first papilla region 108A matches the second papilla region 118A. Here, the registration of the observation target part image 116 with respect to the first duodenum image 108 is performed, but this is realized, for example, by registering a plurality of feature points common between the first papilla region 108A and the second papilla region 118A.

In a state in which the observation target part image 116 is registered with the first duodenum image 108 in this way, the synthesis unit 80E synthesizes the duct image 120 with the first duodenum image 108 to generate a synthesis image 123. That is, the synthesis unit 80E synthesizes the duct image 120 with the first duodenum image 108 by assigning the duct image 120 to a position (that is, a position corresponding to the position of the duct image 120 connected to the second papilla region 118A) corresponding to the second papilla region 118A in the first papilla region 108A.

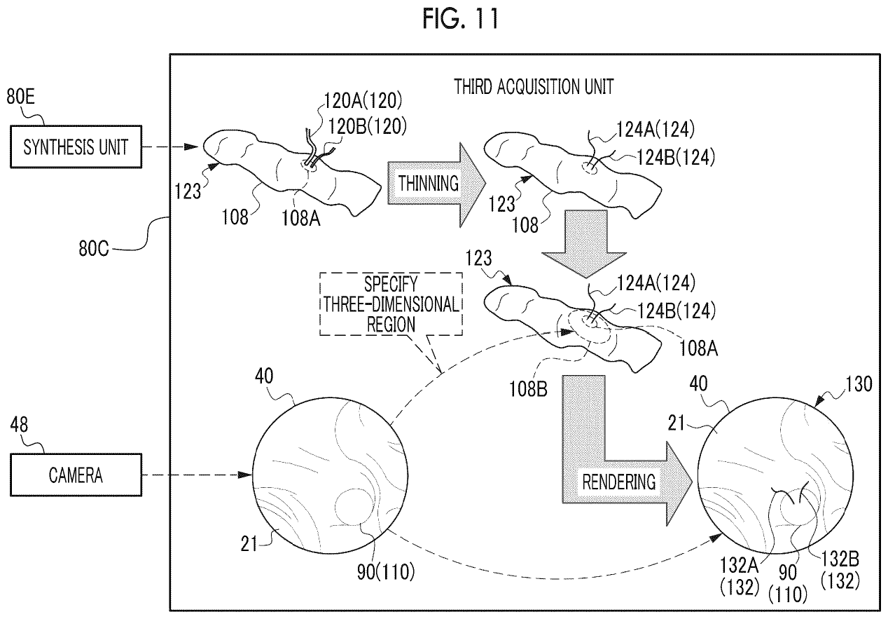

As shown in FIG. 11 as an example, the third acquisition unit 80C acquires a three-dimensional running direction image 124 as information related to the running direction 94 of the duct 92 leading to the duodenum 88 (see FIG. 4) based on the synthesis image 123 generated by the synthesis unit 80E. The three-dimensional running direction image 124 is an image in which the running direction 94 of the duct 92 is represented by a three-dimensional image. The three-dimensional running direction image 124 is an example of the “direction information” and the “three-dimensional direction image” according to the technology of the present disclosure.

The third acquisition unit 80C performs thinning processing on the duct image 120 synthesized with the first duodenum image 108 to generate a three-dimensional running direction image 124 (for example, a curve indicating the running direction 94) indicating the running direction 94 of the duct 92 indicated by the duct image 120. The running direction 94 of the duct 92 can also be referred to as an axial direction of the duct image 120.

The three-dimensional running direction image 124 includes a three-dimensional bile duct direction image 124A and a three-dimensional pancreatic duct direction image 124B. The three-dimensional bile duct direction image 124A shows the running direction 94A (see FIG. 4) of the bile duct 92A, and is obtained by thinning the bile duct image 120A. The three-dimensional pancreatic duct direction image 124B shows the running direction 94B (see FIG. 4) of the pancreatic duct 92B, and is obtained by thinning the pancreatic duct image 120B.

The third acquisition unit 80C acquires the captured image 40 including the papilla image 110 from the camera 48. The third acquisition unit 80C specifies a three-dimensional region 108B corresponding to the captured image 40 acquired from the camera 48 from the synthesis image 123. The captured image 40 acquired from the camera 48 by the third acquisition unit 80C corresponds to a two-dimensional image in which the three-dimensional region 108B is projected onto a plane. The third acquisition unit 80C sets the captured image 40 as a projection surface at a position directly facing the three-dimensional region 108B in the synthesis image 123. The third acquisition unit 80C generates a synthesized captured image 130 by rendering the three-dimensional running direction image 124 in the captured image 40 set in the synthesis image 123.

The synthesized captured image 130 is an image (for example, an image in which a two-dimensional running direction image 132 is superimposed on the captured image 40) including the captured image 40 and the two-dimensional running direction image 132. The two-dimensional running direction image 132 is an image (that is, an image in which the three-dimensional running direction image 124 is projected onto the captured image 40) in which the three-dimensional running direction image 124 is rendered on the captured image 40. The two-dimensional running direction image 132 includes a two-dimensional bile duct direction image 132A and a two-dimensional pancreatic duct direction image 132B. The two-dimensional bile duct direction image 132A is an image in which the three-dimensional bile duct direction image 124A is rendered on the captured image 40. The two-dimensional pancreatic duct direction image 132B is an image in which the three-dimensional pancreatic duct direction image 124B is rendered on the captured image 40.

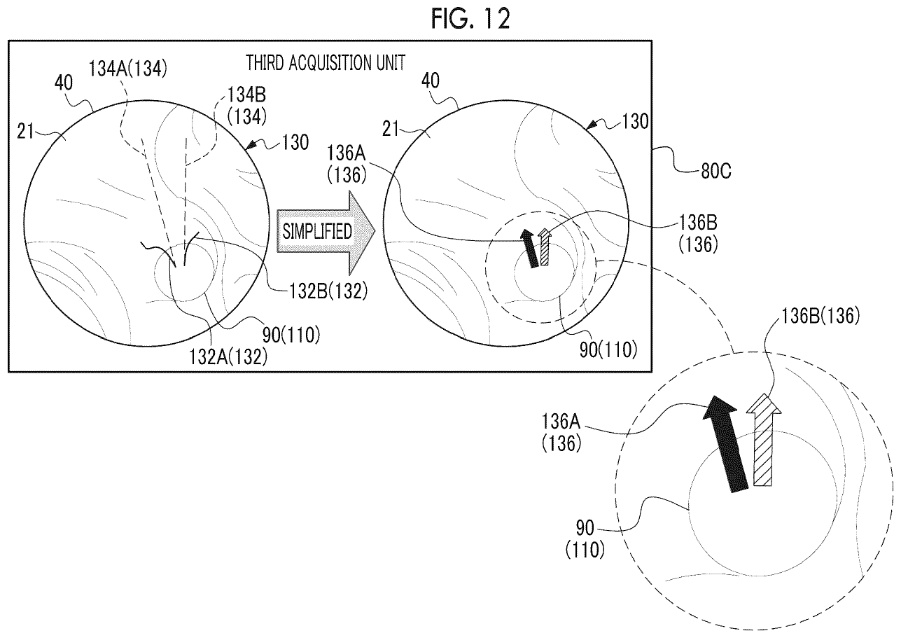

As shown in FIG. 12 as an example, the third acquisition unit 80C acquires a simplified running direction image 136 obtained by simplifying the two-dimensional running direction image 132 in the synthesized captured image 130. The simplified running direction image 136 is an example of the “running direction image” according to the technology of the present disclosure.

In order to generate the simplified running direction image 136, first, the third acquisition unit 80C extracts a tangent line 134 from the two-dimensional running direction image 132. The tangent line 134 is a tangent line with respect to an end (that is, a position where the opening 90A1 (see FIG. 4) is captured) on the papilla image 110 side in the two-dimensional running direction image 132. The tangent line 134 is classified into a bile duct tangent line 134A and a pancreatic duct tangent line 134B. The bile duct tangent line 134A is a tangent line with respect to an end (that is, a position at which the opening 90A1 leading to the bile duct 92A (see FIG. 4) is captured) on the papilla image 110 side in the two-dimensional bile duct direction image 132A. The pancreatic duct tangent line 134B is a tangent line with respect to an end (that is, a position at which the opening 90A1 leading to the pancreatic duct 92B (see FIG. 4) is captured) on the papilla image 110 side in the two-dimensional pancreatic duct direction image 132B.

The third acquisition unit 80C generates a simplified running direction image 136 by imaging the running direction 94 of the duct 92 in accordance with the synthesized captured image 130 based on the two-dimensional running direction image 132. For example, the third acquisition unit 80C generates the simplified running direction image 136 by imaging the tangent line 134. The simplified running direction image 136 is an image showing an arrow formed along the tangent line 134 with an end (that is, a position at which the opening 90A1 leading to the duct 92 (see FIG. 4) is captured) on the papilla image 110 side of the tangent line 134 as a start point. The direction indicated by the arrow shown in the simplified running direction image 136 corresponds to the running direction 94 of the duct 92 at the position of the opening 90A1.

In the present embodiment, the start point of the arrow indicated by the simplified running direction image 136 is an example of the “base point” according to the technology of the present disclosure, the running direction 94 of the duct 92 is an example of the “first direction” and the “direction corresponding to the insertion direction” according to the technology of the present disclosure, and the simplified running direction image 136 is an example of the “image indicating the first direction” according to the technology of the present disclosure.

The simplified running direction image 136 includes a simplified bile duct direction image 136A and a simplified pancreatic duct direction image 136B. The simplified bile duct direction image 136A is an image showing an arrow formed along the bile duct tangent line 134A with an end (that is, a position at which the opening 90A1 leading to the bile duct 92A (see FIG. 4) is captured) on the papilla image 110 side of the bile duct tangent line 134A as a start point. The direction indicated by the arrow shown in the simplified bile duct direction image 136A corresponds to the running direction 94A (see FIG. 4) of the bile duct 92A at the position of the opening 90A1, and is referred to by the doctor 14 as the insertion direction of the cannula 54A or the like with respect to the bile duct 92A. The insertion direction of the cannula 54A or the like with respect to the bile duct 92A is an example of the “insertion direction of a medical instrument to be inserted into a duct” according to the technology of the present disclosure.

The simplified pancreatic duct direction image 136B is an image showing an arrow formed along the pancreatic duct tangent line 134B with an end (that is, a position at which the opening 90A1 leading to the pancreatic duct 92B (see FIG. 4) is captured) on the papilla image 110 side of the pancreatic duct tangent line 134B as a start point. The direction indicated by the arrow shown in the simplified pancreatic duct direction image 136B corresponds to the running direction 94B (see FIG. 4) of the pancreatic duct 92B at the position of the opening 90A1, and is referred to by the doctor 14 as the insertion direction of the cannula 54A or the like with respect to the pancreatic duct 92B. The insertion direction of the cannula 54A or the like with respect to the pancreatic duct 92B is an example of the “insertion direction of a medical instrument to be inserted into a duct” according to the technology of the present disclosure.

The simplified bile duct direction image 136A and the simplified pancreatic duct direction image 136B are generated in a distinguishable manner. For example, the third acquisition unit 80C generates the simplified bile duct direction image 136A and the simplified pancreatic duct direction image 136B in a distinguishable manner by changing the color, density, brightness, and/or pattern of the simplified bile duct direction image 136A and the simplified pancreatic duct direction image 136B.

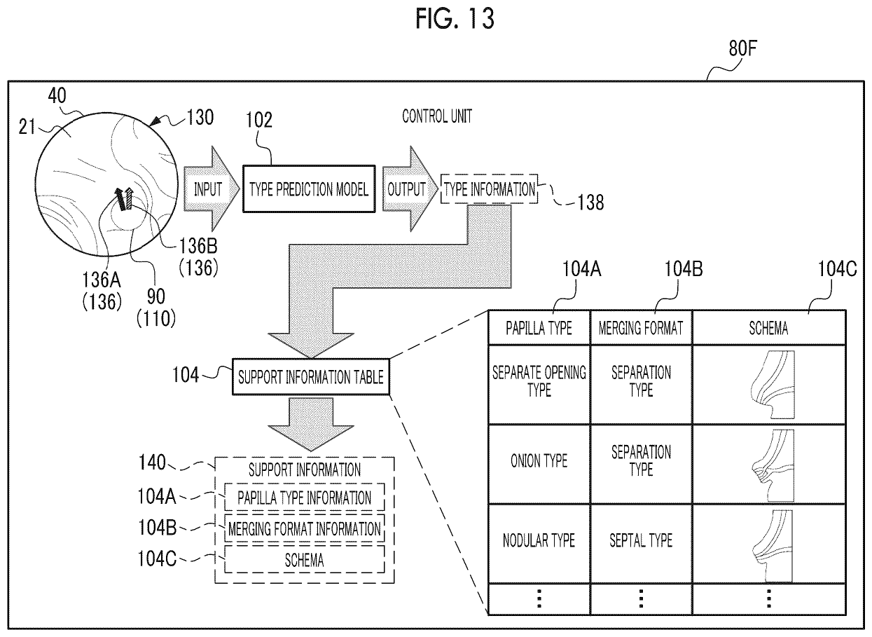

As shown in FIG. 13 as an example, the control unit 80F predicts the type of the papilla 90 captured in the synthesized captured image 130 by performing image recognition processing using the type prediction model 102 on the synthesized captured image 130 acquired by the third acquisition unit 80C. Then, the control unit 80F acquires type information 138 (for example, the name of the type of the papilla 90) indicating a predicted type.

The type prediction model 102 is obtained by performing machine learning using second training data on the neural network to optimize the neural network. The second training data is a plurality of data items (that is, data corresponding to a plurality of frames) in which second example data and second correct answer data have been associated with each other. The second example data is, for example, an image (for example, an image corresponding to the synthesized captured image 130 to which the simplified running direction image 136 is added) obtained by assigning an image corresponding to the simplified running direction image 136 to an image obtained by imaging a part (for example, an inner wall of the duodenum) that can be a target for the ERCP examination. The second correct answer data refers to correct answer data (that is, an annotation) corresponding to the second example data. An example of the second correct answer data is an annotation indicating the type of the papilla 90.

In addition, here, although the image obtained by assigning the image corresponding to the simplified running direction image 136 to the image obtained by imaging a part that can be a target for the ERCP examination is shown as example data included in the second training data, this is merely an example. For example, the example data included in the second training data may be an image itself (that is, an image corresponding to the captured image 40 to which the simplified running direction image 136 is not assigned) obtained by imaging a part that can be a target for the ERCP examination.

In addition, here, although the form example in which only one type prediction model 102 is used by the control unit 80F is described, this is merely an example. For example, the type prediction model 102 selected from a plurality of type prediction models 102 may be used by the control unit 80F. In this case, various type prediction models 102 are created by performing machine learning specialized for each procedure (for example, the position of the duodenoscope 12 with respect to the papilla 90) of the ERCP examination, and the type prediction model 102 corresponding to the procedure of the ERCP examination currently being performed may be selected and used by the control unit 80F.

The control unit 80F inputs the synthesized captured image 130 acquired by the third acquisition unit 80C to the type prediction model 102. Accordingly, the type prediction model 102 predicts the type of the papilla 90 included in the input synthesized captured image 130 and outputs type information 138 indicating a predicted type. The control unit 80F acquires the type information 138 output from the type prediction model 102.

The control unit 80F derives support information 140, corresponding to the type information 138 acquired from the type prediction model 102, from the support information table 104. The support information table 104 is a table in which papilla type information 104A, merging format information 104B, and a schema 104C that have a correspondence relationship with each other are associated with each other. The papilla type information 104A is information (for example, the name of the type of the papilla 90) capable of specifying the type of the papilla 90. The merging format information 104B is determined for each type of the papilla 90, and is information for specifying a merging format in which the bile duct and the pancreatic duct merge with each other. The schema 104C is a schematic diagram schematically showing an aspect in which the bile duct and the pancreatic duct merge with each other.

The support information 140 is information including the papilla type information 104A, the merging format information 104B, and the schema 104C corresponding to the type information 138 acquired from the type prediction model 102 by the control unit 80F.

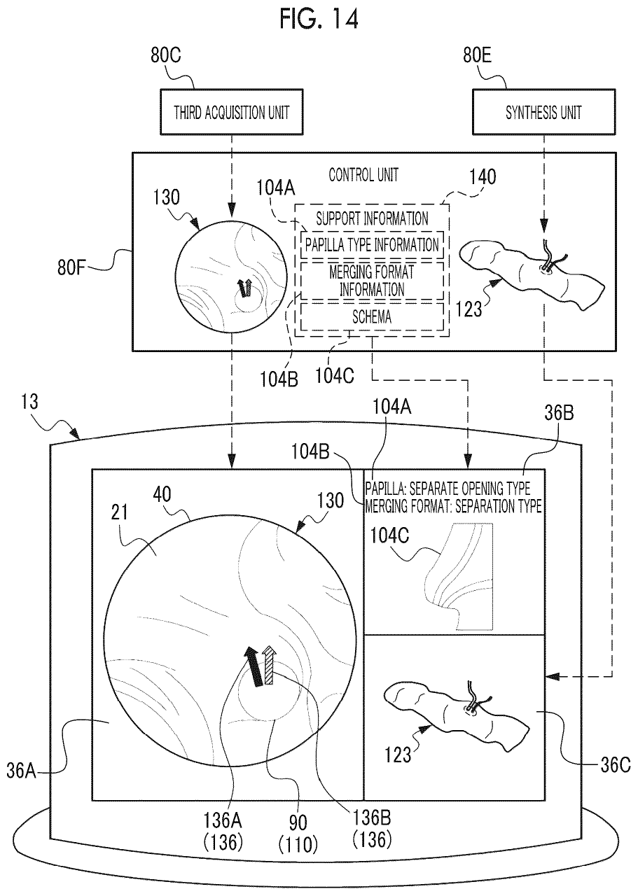

As shown in FIG. 14 as an example, the control unit 80F displays the synthesized captured image 130 acquired by the third acquisition unit 80C on the screen 36A, displays the support information 140 on the screen 36B, and displays the synthesis image 123 generated by the synthesis unit 80E on the screen 36C. The synthesized captured image 130 displayed on the screen 36A is updated in real time. In this case, for example, the synthesis image 123 is updated each time the captured image 40 is acquired by the first acquisition unit 80A and the time-series image group 106 is updated, and the synthesized captured image 130 including the two-dimensional running direction image 132 is updated and the simplified running direction image 136 is also updated accordingly each time the synthesis image 123 is updated.

The update of the synthesized captured image 130 displayed on the screen 36A may be performed each time the first acquisition unit 80A acquires the captured image 40 in units of one frame, or may be performed each time the first acquisition unit 80A acquires the captured image 40 in units of a plurality of frames.

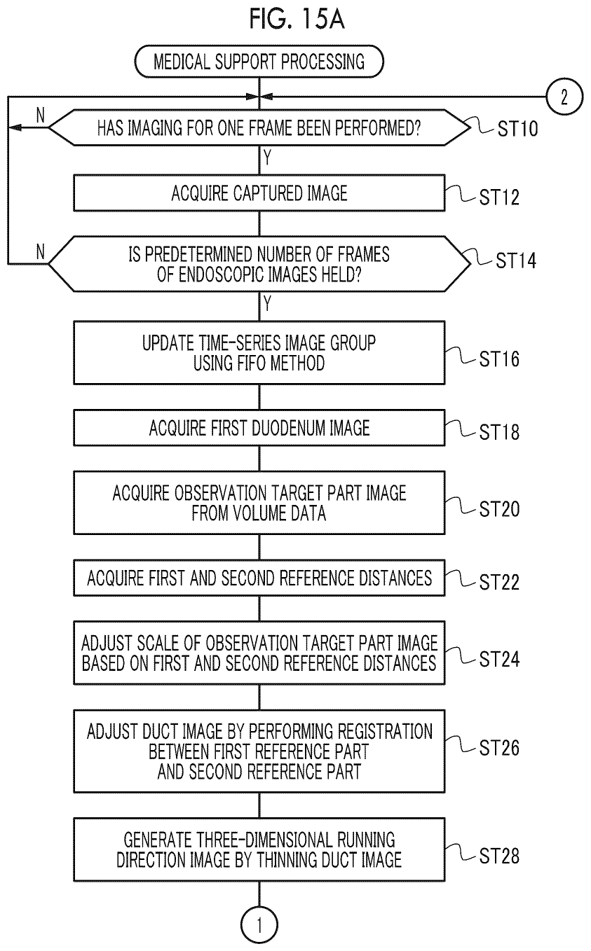

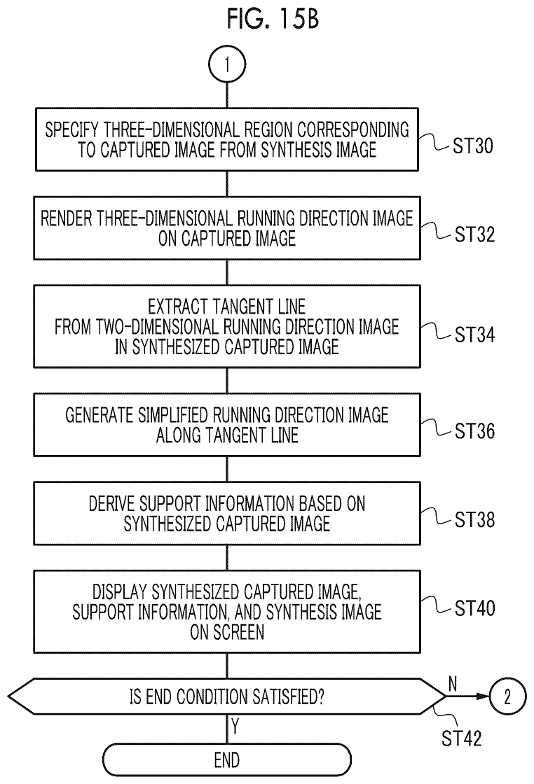

Next, the operation of the portion of the duodenoscope system 10 according to the technology of the present disclosure will be described with reference to FIGS. 15A and 15B.

FIGS. 15A and 15B show an example of a flow of the medical support processing executed by the processor 80. The flow of the medical support processing shown in FIGS. 15A and 15B is an example of the “medical support method” according to the technology of the present disclosure.

In the medical support processing shown in FIG. 15A, first, in step ST10, the first acquisition unit 80A determines whether or not one frame of imaging for the observation target 21 is performed by the camera 48. In step ST10, in a case where the camera 48 does not perform one frame of imaging for the observation target 21, the determination result is “No”, and the determination in step ST10 is performed again. In step ST10, in a case in which the camera 48 performs one frame of imaging for the observation target 21, the determination result is “Yes”, and the medical support processing proceeds to step ST12.

In step ST12, the first acquisition unit 80A acquires one frame of the captured image 40 obtained by imaging the observation target 21 with the camera 48 (see FIG. 6). After the processing in step ST12 is executed, the medical support processing proceeds to step ST14.

In step ST14, the first acquisition unit 80A determines whether or not a predetermined number of frames of the captured images 40 is held. In a case where the predetermined number of frames of captured images 40 is not held in step ST14, the determination result is “No”, and the medical support processing proceeds to step ST10. In a case where the predetermined number of frames of captured images 40 is held in step ST14, the determination result is “Yes”, and the medical support processing proceeds to step ST16.

In step ST16, the first acquisition unit 80A updates the time-series image group 106 by adding the captured image 40 acquired in step ST12 to the time-series image group 106 using the FIFO method (see FIG. 6). After the processing in step ST16 is executed, the medical support processing proceeds to step ST18.

In step ST18, the first acquisition unit 80A acquires the first duodenum image 108 by inputting the time-series image group 106 to the 3D construction model 98. After the processing in step ST18 is executed, the medical support processing proceeds to step ST20.

In step ST20, the second acquisition unit 80B acquires the observation target part image 116 from the volume data 112 (see FIG. 7). After the processing in step ST20 is executed, the medical support processing proceeds to step ST22.

In step ST22, the adjustment unit 80D acquires the first reference distance 126 (see FIG. 8) based on the captured image 40 used to acquire the first duodenum image 108 and on a distance measurement result obtained by the distance-measuring sensor 49, and acquires the second reference distance 128 (see FIG. 8) using the second duodenum image 118 included in the observation target part image 116 acquired by the second acquisition unit. After the processing in step ST22 is executed, the medical support processing proceeds to step ST24.

In step ST24, the adjustment unit 80D adjusts the scale of the observation target part image 116 acquired in step ST20 based on the difference degree 129 between the first reference distance 126 and the second reference distance 128 acquired in step ST22 (see FIG. 9). After the processing in step ST24 is executed, the medical support processing proceeds to step ST26.

In step ST26, the synthesis unit 80E adjusts the duct image 120 by matching the first papilla region 108A included in the first duodenum image 108 acquired in step ST18 with the second papilla region 118A included in the observation target part image 116 of which the scale is adjusted in step ST24. The synthesis unit 80E generates the synthesis image 123 by synthesizing the duct image 120 with the first duodenum image 108 (see FIG. 10). After the processing in step ST26 is executed, the medical support processing proceeds to step ST28.

In step ST28, the thinning processing is performed on the duct image 120 to generate the three-dimensional running direction image 124 indicating the running direction 94 of the duct 92 indicated by the duct image 120 (see FIG. 11). After the processing in step ST28 is executed, the medical support processing proceeds to step ST30 shown in FIG. 15B.

In step ST30 shown in FIG. 15B, the third acquisition unit 80C acquires the captured image 40 including the papilla image 110 from the camera 48 (refer to FIG. 11). Then, the third acquisition unit 80C specifies the three-dimensional region 108B, corresponding to the captured image 40 acquired from the camera 48, from the synthesis image 123 (see FIG. 11). After the process in step ST30 is executed, the medical support processing proceeds to step ST32.

In step ST32, the third acquisition unit 80C sets the captured image 40 as the projection surface at a position directly facing the three-dimensional region 108B in the synthesis image 123. Then, the third acquisition unit 80C renders the three-dimensional running direction image 124 on the captured image 40 set in the synthesis image 123 to generate the synthesized captured image 130 (refer to FIG. 11).

After the processing in step ST32 is executed, the medical support processing proceeds to step ST34.

In step ST34, the third acquisition unit 80C extracts the tangent line 134 from the two-dimensional running direction image 132 included in the synthesized captured image 130 (see FIG. 12). After the process in step ST34 is executed, the medical support processing proceeds to step ST36.

In step ST36, the third acquisition unit 80C generates the simplified running direction image 136 along the tangent line 134 based on the tangent line 134 (see FIG. 12). After the process in step ST36 is executed, the medical support processing proceeds to step ST38.

In step ST38, the control unit 80F derives the support information 140 based on the synthesized captured image 130 (refer to FIG. 13). After the processing in step ST38 is executed, the medical support processing proceeds to step ST40.

In step ST40, the control unit 80F displays the synthesized captured image 130 generated in step ST32 on the screen 36A, displays the support information 140 derived in step ST38 on the screen 36B, and displays the synthesis image 123 generated in step ST26 on the screen 36C. After the processing in step ST40 is executed, the medical support processing proceeds to step ST42.

In step ST42, the control unit 80F determines whether or not a medical support processing end condition is satisfied. An example of the medical support processing end condition is a condition (for example, a condition in which an instruction to end the medical support processing is received by the receiving device 62) in which an instruction to end the medical support processing is issued to the duodenoscope system 10.

In step ST42, in a case in which the medical support processing end condition is not satisfied, the determination result is “No”, and the medical support processing proceeds to step ST10 shown in FIG. 15A. In a case in which the medical support processing end condition is satisfied in step ST42, an affirmative determination is made, and the medical support processing ends.

As described above, in the duodenoscope system 10, the first duodenum image 108 is generated based on the captured image 40, and the first papilla region 108A is acquired from the first duodenum image 108. In addition, the second papilla region 118A and the three-dimensional running direction image 124 are acquired from the volume data 112. In addition, the three-dimensional running direction image 124 adjusted by matching the first papilla region 108A with the second papilla region 118A is imaged in accordance with the captured image 40. Accordingly, the simplified running direction image 136 is generated (see FIG. 12). Then, the synthesized captured image 130 is displayed on the screen 36A. The simplified running direction image 136 is displayed in the synthesized captured image 130 (FIG. 14). The direction indicated by the arrow shown in the simplified running direction image 136 corresponds to the running direction 94 of the duct 92 at the position of the opening 90A1. Therefore, the doctor 14 who observes the synthesized captured image 130 can be made to visually recognize the running direction 94 of the duct 92 leading to the duodenum 88 through the synthesized captured image 130.

In addition, in the duodenoscope system 10, the duct 92 includes the bile duct 92A and the pancreatic duct 92B. Therefore, the doctor 14 who observes the synthesized captured image 130 can be made to visually recognize the running direction 94A of the bile duct 92A leading to the duodenum 88 and the running direction 94B of the pancreatic duct 92B leading to the duodenum 88 through the synthesized captured image 130.

In addition, in the duodenoscope system 10, the position of the three-dimensional running direction image 124 is adjusted by performing the registration between the first papilla region 108A and the second papilla region 118A. Both the first papilla region 108A and the second papilla region 118A are image regions indicating the papilla 90. Therefore, the first papilla region 108A and the second papilla region 118A can be easily registered. As a result, the position of the three-dimensional running direction image 124 can be easily adjusted.

In addition, in the duodenoscope system 10, the simplified running direction image 136 is generated based on the three-dimensional running direction image 124 adjusted by performing the registration between the first papilla region 108A and the second papilla region 118A. The first papilla region 108A is an image region obtained from the first duodenum image 108 generated based on the captured image 40, and the second papilla region 118A is an image region obtained from the observation target part image 116 acquired from the volume data 112. Accordingly, the first papilla region 108A and the second papilla region 118A used for generating the simplified running direction image 136 can be easily obtained. As a result, the simplified running direction image 136 can be easily generated.

In addition, in the duodenoscope system 10, the first duodenum image 108 is generated based on the updated captured image 40 each time the captured image 40 is updated in units of one frame. In addition, the simplified running direction image 136 is generated based on the three-dimensional running direction image 124 each time the first duodenum image 108 is generated. Then, the synthesized captured image 130 based on the updated captured image 40 is displayed on the screen 36A, and each time the simplified running direction image 136 is generated, the generated simplified running direction image 136 is displayed in the synthesized captured image 130 (see FIG. 14). Therefore, each time the captured image 40 is updated, the doctor 14 who observes the synthesized captured image 130, which is generated based on the captured image 40, can be made to visually recognize the running direction 94 of the duct 92 leading to the duodenum 88 through the synthesized captured image 130.

In addition, in the duodenoscope system 10, the simplified running direction image 136 displayed in the synthesized captured image 130 is updated in real time. Therefore, the doctor 14 who observes the synthesized captured image 130 can be made to visually recognize the current running direction 94 of the duct 92, corresponding to the synthesized captured image 130 displayed on the screen 36A, through the synthesized captured image 130.

In addition, in the duodenoscope system 10, an image in which the three-dimensional running direction image 124 is rendered on the captured image 40 set as a projection surface at a position directly facing the three-dimensional region 108B in the synthesis image 123 is used as the simplified running direction image 136. Therefore, the running direction 94 of the duct 92 can be displayed as a two-dimensional image in the captured image 40.

In addition, in the duodenoscope system 10, the simplified running direction image 136 is displayed on the screen 36A along the tangent line 134. The tangent line 134 is a tangent line with respect to the duct 92 at the opening 90A1 of the papilla 90, and is associated with the papilla image 110 showing the papilla 90. Therefore, the simplified running direction image 136 is displayed on the screen 36A in association with the papilla image 110. Therefore, the doctor 14 who observes the synthesized captured image 130 can be made to visually recognize the relationship (for example, the positional relationship) between the running direction 94 of the duct 92 and the papilla 90.

In addition, in the duodenoscope system 10, an image showing the running direction 94 along the duct 92 with the opening 90A1 of the papilla 90 as a start point is displayed in the synthesized captured image 130 as the simplified running direction image 136. Therefore, the doctor 14 who observes the synthesized captured image 130 can be made to visually recognize the running direction 94 along the duct 92 with the opening 90A1 of the papilla 90 as the start point. In addition, since the running direction 94 along the duct 92 with the opening 90A1 of the papilla 90 as a start point is a direction corresponding to the insertion direction of the cannula 54A or the like, it is possible to support the doctor 14 who observes the synthesized captured image 130 in smoothly inserting the cannula 54A or the like into the duct 92.

In addition, in the duodenoscope system 10, the scale of the three-dimensional running direction image 124 is adjusted based on the distance 125 measured by the distance-measuring sensor 49 and on the observation target part image 116 acquired from the volume data 112. Therefore, the two-dimensional running direction image 132 can be generated based on the high-accuracy three-dimensional running direction image 124.

In addition, in the above embodiment, the form example in which the simplified running direction image 136 exemplified as an example of the “running direction image” according to the technology of the present disclosure is displayed on the screen 36A has been described, but this is merely an example. For example, the whole or a part (for example, a part of the image that continues from the opening 90A1 to the back side of the duct 92) of the two-dimensional running direction image 132 may be displayed on the screen 36A instead of the simplified running direction image 136 or together with the simplified running direction image 136.

In the above embodiment, although the form example in which the synthesis unit 80E adjusts the position of the duct image 120 by comparing the observation target part image 116 with the first duodenum image 108 to perform the registration has been described, the technology of the present disclosure is not limited to this. For example, the synthesis unit 80E may adjust the position of the duct image 120 by performing the registration between the observation target part image 116 and the first duodenum image 108 by using three-dimensional coordinates defining the pixels of the observation target part image 116 and three-dimensional coordinates defining the pixels of the first duodenum image 108. In this case, for example, the registration between the observation target part image 116 and the first duodenum image 108 may be performed based on the distance (that is, the plurality of distances 125 measured by the distance-measuring sensor 49) from the reference position (for example, the position of the pylorus) to the plurality of feature points of the papilla 90, the three-dimensional coordinates of the plurality of feature points in the first duodenum image 108, and the three-dimensional coordinates of the plurality of feature points in the observation target part image 116. The three-dimensional coordinates of the plurality of feature points in the first duodenum image 108 are an example of the “first reference part information” according to the technology of the present disclosure, and the three-dimensional coordinates of the plurality of feature points in the observation target part image 116 are an example of the “second reference part information” according to the technology of the present disclosure.

In the above embodiment, although the form example has been described in which the synthesis unit 80E performs the registration between the first duodenum image 108 and the observation target part image 116 by using the first papilla region 108A and the second papilla region 118A indicating the papilla 90, the technology of the present disclosure is not limited to this. For example, the synthesis unit 80E may perform the registration between the first duodenum image 108 and the observation target part image 116 by using information (for example, three-dimensional coordinates or a three-dimensional image) capable of three-dimensionally specifying a medical marker (for example, a hemostatic clip or the like) included in the duodenum 88 and/or information (for example, three-dimensional coordinates or a three-dimensional image) capable of three-dimensionally specifying the fold of the duodenum 88. In this case as well, the same effects as those of the above embodiment can be obtained.

In addition, here, as an example of the “first reference part information” according to the technology of the present disclosure, the information capable of three-dimensionally specifying the papilla 90, the information capable of three-dimensionally specifying the medical marker, and/or the information capable of three-dimensionally specifying the fold are shown. However, the information capable of three-dimensionally specifying the reference part to be used for the registration may be determined according to the instruction (for example, the instruction received by the receiving device 62) given by the doctor 14 who observes the captured image 40 displayed on the screen 36A. In addition, the front end position (that is, a position in contact with the opening 90A1) of the treatment tool 54 may be detected by performing the image recognition processing using the AI method or a non-AI method according to the instruction (for example, the instruction received by the receiving device 62) given by the doctor 14 in a case where the treatment tool 54 has reached the opening 90A1, and the information capable of three-dimensionally specifying the detected front end position may be used as the information capable of three-dimensionally specifying the papilla 90.

In the above embodiment, the second reference distance 128 is exemplified as an example of the “distance from the endoscope scope to the intestinal wall” according to the technology of the present disclosure, and the form example in which the distance from the pylorus to the papilla 90 is measured as the second reference distance 128 by using the second duodenum image 118 has been described, but this is merely an example. For example, the second reference distance 128 may be measured by using positional information (that is, information indicating the current position of the camera 48) and posture information (that is, information indicating the posture of the camera 48), which are obtained by using an optical fiber sensor provided inside the duodenoscope body 18 (for example, the insertion part 44 and the distal end part 46) in a longitudinal direction. In addition, the second reference distance 128 may be measured using a known electromagnetic navigation method in the related art, without being limited to such an optical fiber method.

In the above embodiment, the second reference distance 128 is exemplified as an example of the “distance from the endoscope scope to the intestinal wall” according to the technology of the present disclosure, and the distance from the pylorus to the papilla 90 is exemplified as an example of the second reference distance 128, but these are merely examples. Other examples of the distance from the camera 48 to the intestinal wall 88A include a distance from the distal end of the distal end part 16 to the intestinal wall 88A and a distance from the imaging position (that is, the current position of the camera 48) of the camera 48 to the intestinal wall 88A.

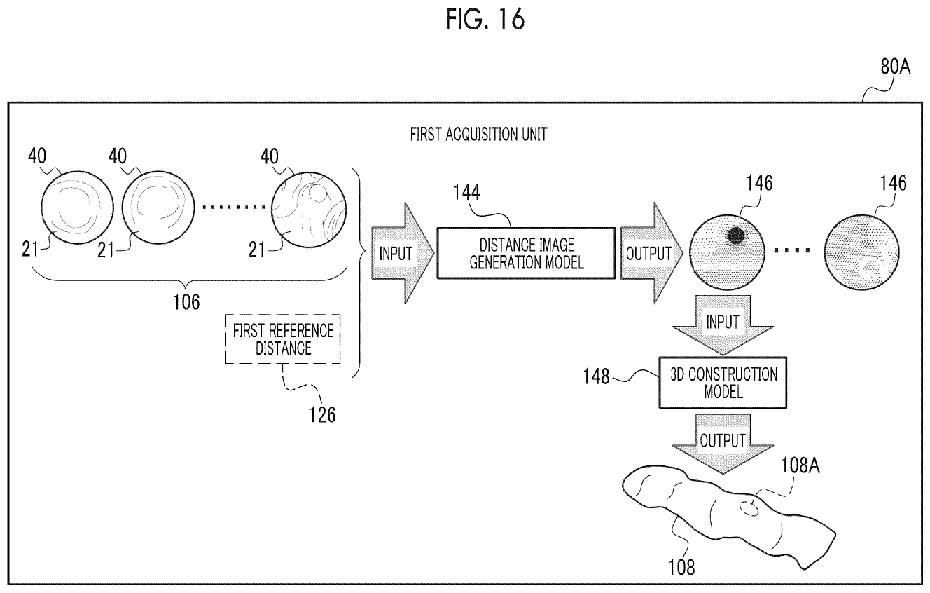

In the above embodiment, the form example (see FIG. 6) in which the first duodenum image 108 is generated based on the time-series image group 106 has been described, but this is merely an example. For example, as shown in FIG. 16, the first acquisition unit 80A may generate the first duodenum image 108 based on a plurality of distance images 146. In this case, for example, the first acquisition unit 80A acquires the plurality of distance images 146 using a distance image generation model 144. The acquisition of the plurality of distance images 146 is realized, for example, by generating the plurality of distance images 146 by using the 3D construction model 98.

The distance image generation model 144 is a generation model using a neural network, and generates the plurality of distance images 146 based on the time-series image group 106 and the first reference distance 126. The distance image generation model 144 is a trained model obtained by performing machine learning on a neural network using a plurality of images corresponding to the time-series image group 106 and a distance corresponding to the first reference distance 126 as training data. An example of the distance image generation model 144 is an auto-encoder such as a VAE or a GAN.

The first acquisition unit 80A inputs the time-series image group 106 and the first reference distance 126 to the distance image generation model 144. Accordingly, the distance image generation model 144 generates and outputs the plurality of distance images 146 based on the time-series image group 106 and the first reference distance 126. The first acquisition unit 80A acquires the plurality of distance images 146 output from the distance image generation model 144.

The first acquisition unit 80A generates the first duodenum image 108 by using a 3D construction model 148. The 3D construction model 148 is a generation model using a neural network, and generates the first duodenum image 108 based on the plurality of distance images 146. The 3D construction model 148 is different from the 3D construction model 98 shown in FIG. 6 in that the 3D construction model 148 is a trained model obtained by performing machine learning on the neural network using a plurality of distance images corresponding to the plurality of distance images 146 instead of the plurality of images corresponding to the time-series image group 106 as the training data.

The first acquisition unit 80A inputs the plurality of distance images 146 to the 3D construction model 148. In response to this, the 3D construction model 148 generates and outputs the first duodenum image 108 based on the plurality of distance images 146. The first acquisition unit 80A acquires the first duodenum image 108 output from the 3D construction model 148. In the duodenoscope system 10, the first duodenum image 108 acquired by the first acquisition unit 80A is used in the same manner as in the above embodiment. Accordingly, the same effects as in the above embodiment are obtained.

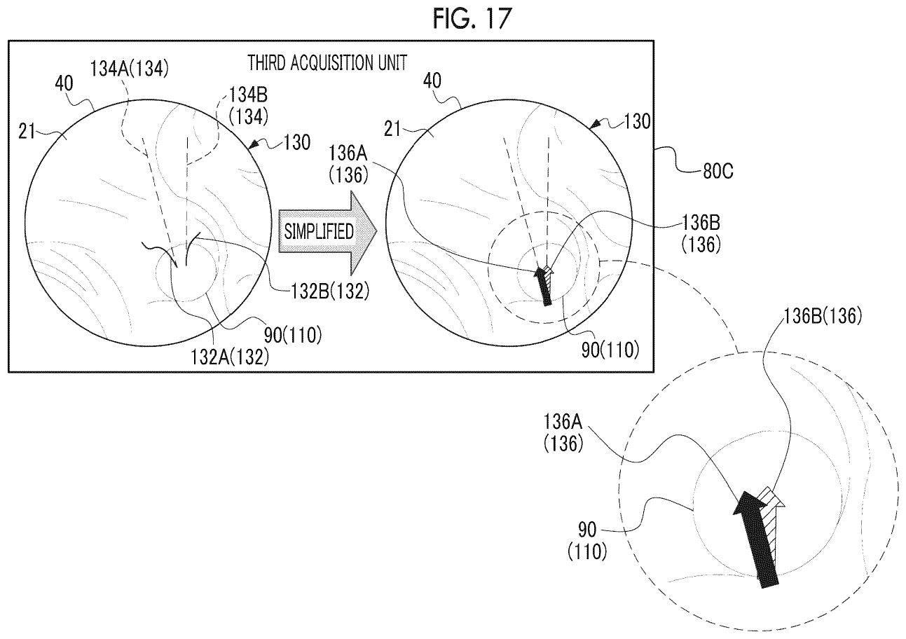

In the above embodiment, although the form example in which the third acquisition unit 80C forms the simplified running direction image 136 along the tangent line 134 with an end (that is, a position at which the opening 90A1 leading to the duct 92 (see FIG. 4) is captured) on the papilla image 110 side of the tangent line 134 as a start point has been described, the technology of the present disclosure is not limited to this. For example, as shown in FIG. 17, the third acquisition unit 80C may form the simplified running direction image 136 along an extension line of the tangent line 134.

In the example shown in FIG. 17, the simplified running direction image 136 is disposed such that the distal end of the arrow indicated by the simplified running direction image 136 is positioned at the end (that is, a position where the opening 90A1 leading to the duct 92 (see FIG. 4) is captured) on the papilla image 110 side of the tangent line 134. Specifically, the simplified bile duct direction image 136A is formed along an extension line of the bile duct tangent line 134A such that the arrow indicated by the simplified bile duct direction image 136A indicates an end (that is, a position at which the opening 90A1 leading to the bile duct 92A (see FIG. 4) is captured) on the papilla image 110 side of the bile duct tangent line 134A. In addition, the simplified pancreatic duct direction image 136B is formed along an extension line of the pancreatic duct tangent line 134B such that the arrow indicated by the simplified pancreatic duct direction image 136B indicates an end (that is, a position where the opening 90A1 leading to the pancreatic duct 92B (see FIG. 4) is captured) on the papilla image 110 side of the pancreatic duct tangent line 134B.

In this way, since the position (that is, the position of the distal end of the arrow shown in the simplified bile duct direction image 136A) indicated by the arrow shown in the simplified bile duct direction image 136A corresponds to the position where the opening 90A1 of the bile duct 92A is present, the doctor 14 who observes the synthesized captured image 130 can be made to visually grasp the position of the opening 90A1 of the bile duct 92A and the insertion direction of the cannula 54A or the like with respect to the opening 90A1 of the bile duct 92A. In addition, since the position (that is, the position of the distal end of the arrow shown in the simplified bile duct direction image 136B) indicated by the arrow shown in the pancreatic duct-bile duct direction image 136B corresponds to the position where the opening 90A1 of the pancreatic duct 92B is present, the doctor 14 who observes the synthesized captured image 130 can be made to visually grasp the position of the opening 90A1 of the pancreatic duct 92B and the insertion direction of the cannula 54 or the like with respect to the opening 90A1 of the pancreatic duct 92B.

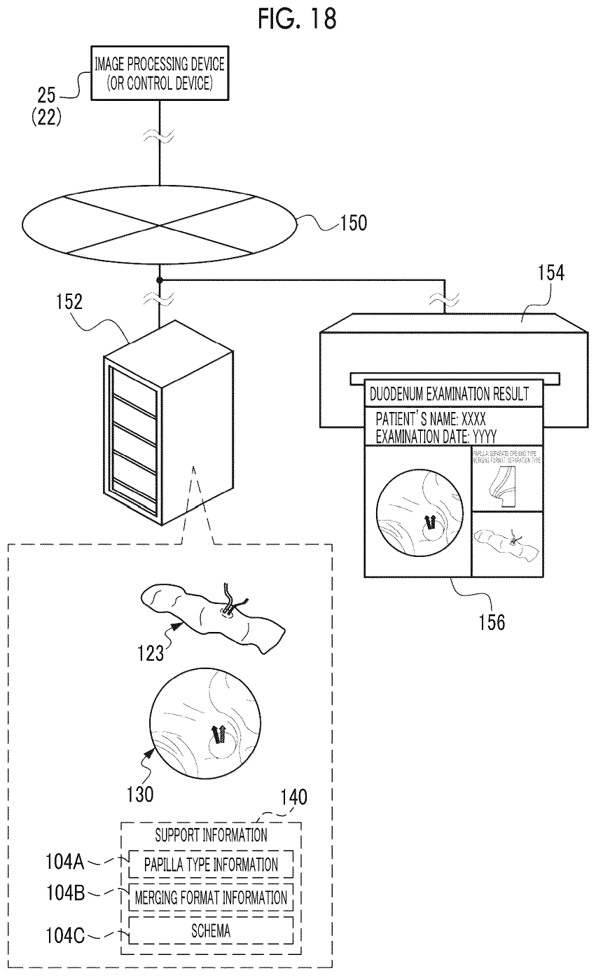

In the above embodiment, although the form example in which the synthesis image 123, the synthesized captured image 130, and the support information 140 are displayed on the display device 13 has been described, the technology of the present disclosure is not limited to this. For example, as shown in FIG. 18, in a case where the image processing device 25 or the control device 22 is connected to a server 152 (for example, a server that manages an electronic medical record) via a network 150, the synthesis image 123, the synthesized captured image 130, and/or the support information 140 may be output from the image processing device 25 or the control device 22 to the server 152 and stored in the server 152. In addition, the synthesis image 123, the synthesized captured image 130, and/or the support information 140 may be stored in the electronic medical record.

In addition, in a case where a printer 154 is connected to the network 150, the synthesis image 123, the synthesized captured image 130, and/or the support information 140 may be output to the printer 154 from the image processing device 25 or the control device 22, and the synthesis image 123, the synthesized captured image 130, and the support information 140 may be printed on a recording medium (for example, paper or the like) by the printer 154.

In the above embodiment, although the form example in which the papilla image 110 is detected by executing the processing (that is, the processing using the papilla detection model 100) using the AI method has been described, the technology of the present disclosure is not limited to this. The papilla image 110 may be detected by executing the processing (for example, template matching or the like) using the non-AI method. The same applies to the processing using the type prediction model 102 shown in FIG. 13.

In the above embodiment, the form example in which the medical support process is performed by the processor 70 of the computer 76 included in the duodenoscope 12 has been described, but the technology of the present disclosure is not limited to this. The device that performs the medical support processing may be provided outside the duodenoscope 12. An example of the device provided outside the duodenoscope 12 is at least one server and/or at least one personal computer that is communicably connected to the duodenoscope 12. In addition, the medical support processing may be performed in a distributed manner by a plurality of devices.

In the above embodiment, although the form example in which the medical support program 96 is stored in the NVM 74 has been described, the technology of the present disclosure is not limited to this. For example, the medical support program 96 may be stored in a computer-readable non-transitory storage medium such as an SSD or a USB memory. The non-transitory storage medium may be a stationary non-transitory storage medium or a portable non-transitory storage medium. The medical support program 96 stored in the non-transitory storage medium is installed in the computer 76 of the duodenoscope 12. The processor 70 executes the medical support processing according to the medical support program 96.

In addition, the medical support program 96 may be stored in a storage device of, for example, another computer or a server that is connected to the duodenoscope 12 via a network. Then, the medical support program 96 may be downloaded and installed in the computer 76 in response to a request from the duodenoscope 12.

In addition, all of the medical support program 96 does not need to be stored in the storage device of, for example, another computer or the server connected to the duodenoscope 12 or the NVM 74, and a portion of the medical support program 96 may be stored therein.

Various processors described below can be used as the hardware resource for executing the medical support processing. An example of the processor is a CPU which is a general-purpose processor that executes software, that is, a program, to function as the hardware resource performing the medical support processing. In addition, an example of the processor includes a dedicated electronic circuit, which is a processor having a dedicated circuit configuration designed to perform specific processing, such as an FPGA, a PLD, or an ASIC. Any processor has a memory built in or connected to it, and any processor executes the medical support processing by using the memory.

The hardware resource for executing the medical support processing may be configured by one of the various processors or by combining two or more processors of the same type or different types (for example, by combining a plurality of FPGAs or by combining a CPU and an FPGA). The hardware resource for executing the medical support processing may also be one processor.

A first example of the configuration using one processor is a form in which one processor is configured by combining one or more CPUs and software, and the processor functions as the hardware resource for executing the medical support processing. A second example of the configuration is an aspect in which a processor that implements the functions of the entire system including a plurality of hardware resources for performing the medical support processing using one IC chip is used. A representative example of this aspect is an SoC. In this way, the medical support processing is implemented by using one or more of the various processors as the hardware resource.

More specifically, an electric circuit in which circuit elements such as semiconductor elements are synthesized can be used as a hardware structure of the various processors. In addition, the above medical support processing is merely an example. Therefore, it goes without saying that unnecessary steps may be deleted, new steps may be added, and the processing order may be changed within a range that does not deviate from the scope.

The above described contents and shown contents are detailed descriptions of portions relating to the technology of the present disclosure and are merely examples of the technology of the present disclosure. For example, the description of the configuration, the function, the operation, and the effect above are the description of examples of the configuration, the function, the operation, and the effect of the portions according to the technology of the present disclosure. Thus, it goes without saying that unnecessary portions may be deleted, new elements may be added, or replacement may be made to the above described contents and shown contents within a range that does not deviate from the scope of the technology of the present disclosure.

In addition, the description of, for example, common technical knowledge that does not need to be particularly described to enable the implementation of the technology of the present disclosure is omitted in the above described contents and shown contents in order to avoid confusion and to facilitate understanding of the portions relating to the technology of the present disclosure.

In the present specification, “A and/or B” is synonymous with “at least one of A or B”. That is, “A and/or B” may mean only A, only B, or a combination of A and B. In the present specification, the same concept as “A and/or B” also applies to a case in which three or more matters are expressed by association with “and/or”.

All documents, patent applications, and technical standards described in the present specification are incorporated in the present specification by reference in their entirety to the same extent as in a case where the individual documents, patent applications, and technical standards are specifically and individually written to be incorporated by reference.

What is claimed is:

1. A medical support device comprising:
a processor,
wherein the processor is configured to:
acquire first reference part information capable of three-dimensionally specifying a reference part included in a duodenum based on a captured image obtained by imaging an intestinal wall of the duodenum with an endoscope scope;
acquire second reference part information related to the reference part and direction information related to a running direction of a duct leading to the duodenum from volume data;
generate a running direction image in which the running direction is imaged in accordance with the captured image based on the direction information adjusted by matching the first reference part information with the second reference part information;
display the captured image on a screen; and
display the running direction image in the captured image.

2. The medical support device according to claim 1,
wherein the first reference part information is a first image region in which the reference part is represented as a three-dimensional image,
the second reference part information is a second image region in which the reference part is represented as a three-dimensional image, and
the processor is configured to generate the running direction image based on the direction information adjusted by performing registration between the first image region and the second image region.

3. The medical support device according to claim 1,
wherein the processor is configured to:
generate a duodenum image in which the duodenum is represented as a three-dimensional image based on the captured image; and
acquire the first reference part information from the duodenum image.

4. The medical support device according to claim 3,
wherein the duodenum image is a three-dimensional image generated based on a plurality of distance images.

5. The medical support device according to claim 4,
wherein, each time the captured image is updated in units of a designated number of frames,
the processor is configured to:
generate the duodenum image based on the updated captured image;
generate the running direction image based on the direction information each time the duodenum image is generated;
display the updated captured image on the screen; and
display the generated running direction image in the captured image each time the running direction image is generated.

6. The medical support device according to claim 1,
wherein the direction information is a three-dimensional direction image in which the running direction is represented as a three-dimensional image, and
the running direction image is an image based on a two-dimensional image obtained by projecting the three-dimensional direction image onto the captured image.

7. The medical support device according to claim 1,
wherein the captured image includes a papilla image showing a duodenal papilla, and
the running direction image is displayed in association with the papilla image.

8. The medical support device according to claim 7,
wherein the duct leads to an opening in the duodenal papilla, and
the running direction image is an image showing a first direction along the duct with the opening as a base point.

9. The medical support device according to claim 8,
wherein the first direction is a direction corresponding to an insertion direction of a medical instrument to be inserted into the duct.

10. The medical support device according to claim 1,
wherein the reference part is a duodenal papilla, a medical marker, and/or a fold.

11. The medical support device according to claim 1,
wherein the processor is configured to adjust a scale of the direction information based on a distance from the endoscope scope to the intestinal wall and on the volume data.

12. The medical support device according to claim 1,
wherein the duct is a bile duct and/or a pancreatic duct.

13. The medical support device according to claim 1,
wherein the running direction image displayed in the captured image is updated in real time.

14. An endoscope comprising:
the medical support device according to claim 1; and
the endoscope scope.

15. A medical support method comprising:
acquiring first reference part information capable of three-dimensionally specifying a reference part included in a duodenum based on a captured image obtained by imaging an intestinal wall of the duodenum with an endoscope scope;
acquiring second reference part information related to the reference part and direction information related to a running direction of a duct leading to the duodenum from volume data;
generating a running direction image in which the running direction is imaged in accordance with the captured image based on the direction information adjusted by matching the first reference part information with the second reference part information;
displaying the captured image on a screen; and
displaying the running direction image in the captured image.

16. A non-transitory computer-readable storage medium storing a program executable by a computer to execute processing comprising:
acquiring first reference part information capable of three-dimensionally specifying a reference part included in a duodenum based on a captured image obtained by imaging an intestinal wall of the duodenum with an endoscope scope;
acquiring second reference part information related to the reference part and direction information related to a running direction of a duct leading to the duodenum from volume data;
generating a running direction image in which the running direction is imaged in accordance with the captured image based on the direction information adjusted by matching the first reference part information with the second reference part information;
displaying the captured image on a screen; and
displaying the running direction image in the captured image.
